(19) United States
(12) Patent Application Publication
(10) Pub. No.: US 2005/0132430 A1
(43) Pub. Date: Jun. 16, 2005

(54) Igamete recruitment and developmental competence in mammals by inhibiting the de-nova sterol biosynthesis and/or promoting sterol efflux

(76) Inventor: Mogens Baltsen, Bagsvaerd (DK)

Correspondence Address: FROMMER LAWRENCE & HAUG, 745 FIFTH AVENUE- 10TH FL., NEW YORK, NY, 10151, US

(21) Appl. No.: 10/874,152

(22) Filed: Jun. 21, 2004

Related U.S. Application Data
(63) Continuation-in-part of application No. PCT/DK02/00899, filed on Dec. 20, 2002.

(30) Foreign Application Priority Data
Dec. 21, 2001 (DK) PA 2001 01947

(51) Int. Cl. A01K 67/027; A61K 31/724; A61K 31/225

(52) U.S. Cl. 800/21; 514/58; 514/423; 514/460; 514/548

(57) ABSTRACT

The present invention relates to methods for increasing the developmental competence of at least one mammalian germ cell, gamete, zygote, early embryo, implanted blastocyst and/or embryo by administering a compound which is capable of inhibiting the de novo biosynthesis of sterols and thereby establishing cellular conditions that improve their development and survival. The invention also relates to methods for increasing the sterol efflux prior to fertilisation from at least one mammalian ovary, oocyte, female gamete, or ovary derived cell surrounding an oocyte by administering a compound which is capable of promoting the sterol efflux and thereby reducing the phospholipid/sterol ratio of said cells.

### FIELD OF INVENTION

The present invention relates to methods for increasing the developmental competence of at least one mammalian germ cell, gamete, zygote, early embryo, implanted blastocyst and/or embryo by administrating a compound which is capable of inhibiting the de novo biosynthesis of sterols and thereby establishing cellular conditions that improve their development and survival. The invention also relates to methods for increasing the sterol efflux prior to fertilisation from at least one mammalian ovary, oocyte, female gamete, or ovary derived cell surrounding an oocyte by administrating a compound which is capable of promoting the sterol efflux and thereby reducing the phopholipid/sterol ratio of said cells.

### BACKGROUND OF THE INVENTION

Medical treatments for infertility have come a long way over the past decades offering the possibility of parenthood through medical intervention to otherwise subfertile or infertile couples. However, a large amount of couples gain no effect from the range of infertility treatments known to the person skilled in the art. Thus, the process of selecting a medical practice for fertility treatment can often be a confusing and emotionally draining experience.

Around 40% of all infertility in humans may be attributed to the woman, 40% to the man and in 20% of cases both partners contribute to the problem. Infertility is defined differently depending on the woman's age. The standard definition of infertility is the inability to achieve a pregnancy after one year of unprotected intercourse in couples where the woman is under age 35. In parallel to this fertility treatments display a drop that starts around the age of 34 years of age falling to almost zero in women over 42 unless donor eggs are used. Thus, improved methods for treating infertility are needed since the average maternal age is increasing.

The development of a new mammal ultimately begins with the development of a fertilisable germ cell, the gamete, prone to participate in the process of fertilisatlon. This process by which two highly specialised cells, the spermatozoon and the oocyte, unite to give rise to the zygote, may in turn give rise to a new individual. In preparation before a possible fertilisation, both male and female germ cell lines undergo a number of changes involving the nucleus as well as the cytoplasm. The purpose of these changes is twofold: 1) to reduce the number of chromosomes from homologous pairs, as represented in the somatic cells (diploid), to singles (haploid), and 2) to alter the shape and biochemical characteristics of the germ cells in preparation for fertilisation.

Ontogeny of Ovarian Follicles and Oocytes

The germ cell progenitor in the female is the so-called primary oocyte. Primary oocytes replicate their DNA and enter the prophase of the first meiotic division. At this time cell degeneration begins and many primary oocytes become atretic and die. All surviving primary oocytes, however, have entered the prophase of the first meiotic division and they are now individually surrounded by a layer of somatic epithelial cells: the primordial follicle.

At birth the primary oocytes of humans are meiotically arrested in the prophase instead of proceeding into the metaphase. In mammals, primary oocytes do not resume melosis until puberty is reached (FIG. 1). Only slightly before puberty do a fraction of the primordial follicles develop through a presumed hormone (gonadotrophin, i.e. follicular stimulating hormone, FSH, and luteinizing hormone, LH) independent stage and reach a gonadotrophin dependent stage, from where further development is dependent on a hormonal stimulation. During the following gonadotrophin dependent growth, follicles of most mammals including humans develop a fluid filled cavity, the so-called antrum, and these follicles are from then termed antral or Graafian follicles. Later again a circulating surge of gonadotropins, mainly LH, trigger the oocytes resume meiosis in context with the follicular eruption and ovulation.

Only a certain number of primary follicles initiate growth from the resting pool. A large number of the follicles undergo atresia and die during the development from primordial follicles to the pre-antral stage. Even from that stage the vast majority of the antral follicles undergo atresia and die, resulting in a significant oocyte loss. These selection processes together assure the exclusion of non-viable or mis-developed germ cells. However, the intra-ovarian regulatory mechanisms also determine that only a certain number of follicles reach ovulation. The average number of ovulated ova is species-specific and relate to the reproductive strategy of the species in question and not to the exclusion of the non-viable or mis-developed germ cells. The regimes that assure an adequate number of oocytes are believed to be related to the level of gonadotrophins in circulation and the number of gonadotrophin receptors on the somatic cell compartment in the pre-antral ovarian follicles.

During the so-called follicular phase, where small antral follicles develop into pre-ovulatory follicles, a stimulation of the adult female with exogenous gonadotrophins result in an increased number of viable oocytes retrievable from an increased number of pre-ovulatory follicles. The clinical treatment based on this is still complicated and in many cases with incomplete success.

The number of retrieved, viable oocytes is still often a limiting factor for later conception success, both in human IVF treatments and during livestock breeding. Also, an expressed disadvantage of the hormonal treatment is the possibility of side effects for the women caused by the increased gonadotrophins in circulation, especially LH or the functional equivalent human chorion gonadotrophin, hCG.

Ontogeny of Spermatogenesis

Germ cell progenitors in the male are the spermatogonia. Pre-spermatogonia are surrounded by a somatic cell and enclosed in so-called testis cords prior to or shortly after birth. This represents a major structural similarity of germ cell development in the two sexes. The surrounding cells are called Sertoli-cells and function as nursing cells for the germ cell development in analogy with the granulosa cell nursing of the oocyte.

A major difference between female and male germ lines is the timing and progression of the meiotic cycle. In the male the diploid pre-spermatogonia somehow differentiate into a resting pool of stem spermatogonia and a mitotically active pool of spermatogonla. The division of the latter give rise to A-spermatogonia that still able to divide mitotically and B-sermatogonia that are committed to enter melosis. Male germ line meiosis takes place only after onset of puberty but, unlike the female germ line diplotene arrest, then proceeds with no arrests until four hapilod germ cells are produced from a single B-spermatogonla (FIG. 1). The product of the two melotic divisions is spermatocytes (1′ and 2′) and the secondary spermatocytes differentiate further into spermatides and later into spermatozoa that are released into the lumen of the seminiferous tubuli in the testes.

After release into the lumen, spermatozoa enter the epididymis where a number of biochemical processes takes place that render the spermatozoa capable of undergoing so-called capacitation, which is a prerequisite for the so-called acrosomal process that is necessary during sperm-oocyte fusion and fertilisation. In nature, capacitation takes place in the uterine tract of the female and involves, amongst other possible factors, a loss of cell membrane cholesterol to the surroundings.

The number and quality of the male germ cells is dependent of a number of factors involving hormones, cellular processes, environmental factors and stress. In principle, the number of male germ cells is unlimited because of the existence of stem-spermatogonia that will produce dividing B-spermatogonia and later mature spermatozoa. However, in practice the number of Sertoli-cells in the testes that nurse the maturation of the male germ cells limits the number.

Low numbers of mature spermatozoa causes male sub- and infertility, low quality of the ejaculated spermatozoa, or both. “Low sperm quality” is a broad operational term that may cover genetic, biochemical and morphological components and even environmentally induced factors.

Sterol Biosynthesis

Sterols are important constituents of all the membranes of all metazoans. Cholesterol is the most prevalent sterol and the natural biosynthetic end-point of sterol biosynthesis in mammals. Sterols are defined as substances containing the perhydrocyclopentan-ophenanthrene four-ring system with at least a carbon17 (C17) substituted side-chain and a C3β-hydroxyl substitution (FIG. 4). In the present context the notion of sterols may be limited to structures that concur to these structural requirements and appear as intermediates from lanosterol to cholesterol in a physiological context (see FIG. 2 for examples).

Biosynthesis of sterols in mammals ultimately starts with a carbon source. cetic acid is the precursor for cholesterol biosynthesis de novo. However, this is not synonymous with the contention that acetic acid only will be used for cholesterol biosynthesis, because a number of branches occur on the metabolic pathway of acetic acid, of which the branch that give rise to cholesterol production is one only (see FIG. 3). The biosynthesis magnitude of any of the products depend on the physiological potentiation of the biosynthetic pathway combined with the regulatory pattern that gives some branches premium as opposed to the other branches. Specifically, the conversion of 3-hydroxy-3-methyl-glutaryl into mevalonc acid step has been recognised for many years as the rate limiting step in the biosynthesis of prenylated proteins, dolichol, coenzyme Q, the side-chain of heme a and cholesterol. This biosynthetic step is catalysed by the enzyme 3-hydroxy-3-methyl-glutaryl-coenzyme A-reductase (HMG-CoA-reductase) (FIG. 3). The biosynthetic pathway between acetic acid and cholesterol involves more than 20 different enzymes. HMG-CoA-reductase appears early in this pathway and represents the rate-limiting enzyme in the synthesis of sterols and other down-stream factors and metabolites (Goldstein et al., 1990).

Sterols and Sterol Dynamics in Germ Cells

Sterols are important constituents of bio-membranes and constitute together with phospholipids the vast lipid material in all membrane structures in cells. Sterols appear as free, i.e. non-covalently modified, sterols in membranes but are stored as sterol-esters in sub-cellular compartments. In mammals, cholesterol is the dominant sterol species and the quantitative appearance of this sterol decides important physical qualities of the membrane. Changes in the cholesterol to phospholipid ratio in the plasma membranes affect membrane permeability, membrane transport properties, cell fusogenicity, enzyme activities and membrane fluidity.

Spermatozoa

The mammalian spermatozoa has a unique composition of sterols as evidenced by the finding of desmosterol in rhesus monkey spermatozoa and desmosterol and cholesta-7,24-dien-3β-ol in hamster spermatozoa (Awano et al., 1989) and testicular meiosis activating sterol (T-MAS) in human spermatozoa (Baltsen et al., 1998). Overall, cholesterol seems to be the major sterol in mammalian spermatozoa. However, the non-cholesterol sterols may constitute up to 90% of the total amount of sterols in certain spermatozoa stages of some species (Awano et al., 1989).

During mating, the sperm capacitates during uterine and tubal transport. Physiologically, the acrosome reaction of the spermatozoa is induced by binding to the zona pelucida (egg-shell) on the oocyte prior to the cellular fusion but also by progesterone that is also produced by the newly ovulated cumulus cells of the cumulus-oocyte complex in the oviduct. Spermatozoa capacitate in vitro by exposition to agent that confers cholesterol loss from the membrane, and exogenous cholesterol and desmosterol inhibit the acrosomal responsiveness to progesterone (Cross, 1996). The sterol content or the content of a molecule in equilibrium with sterols is therefore a key parameter during male germ cell development. It has long been known that the ratio between free cholesterol and phospholipids changes during capacitation of the spermatozoa (Davis, 1981). Free cholesterol has been shown to exert an inhibitory action on capacitation as opposed to its esterifled counterpart (Davis, 1980). Operationally it has been confirmed that cholesterol loss from the plasma membrane is vital for the spermatozoa's ability to fertilise eggs. In some species the acrosome reaction of the spermatozoa is preceded by the removal of sterols from the lipid bilayer of the sperm plasma membrane. Experimentally, sterol labelling in spermatozoa decreases significantly during epididymal passage (Lopez et al., 1991). Also, exogeneous cholesterol inhibits capacitation in mouse spermatozoa (Go et al., 1985).

It is generally believed that the spermatozoa, although able to synthesise cholesterol de novo from acetic acid (Gunasegaram et al., 1995), receive cholesterol from supporting tissue (Cross, 1998). However, this statement is not investigated thoroughly and little is known of the actual sterol metabolism in the maturing male germ cell. Long term treatment of rabbits with cholesterol-rich diets led to a lower sperm count and sperm motility as compared to rabbits fed a control diet (Yamamoto et al., 1999). However, long-term treatment with statins in hypercholesterolemic men seems not to affect plasma testosterone, total sperm count, sperm morphology or sperm motility (Bernini et al., 1998). It is therefore uncertain as to whether the sterol delivery or sterol content of cells in the reproductive tract might be affected by systemic treatment with agents that interfere with sterol dynamics.

It has been shown that the sterol content of spermatozoa membrane determines the extent of expression of certain surface protein important for sperm-egg recognition (Benoff et al. 1993a, b). Cholesterol release is also associated with increasing tyrosine phosphorylation of sperm proteins and rise in the intracellular pH, which both are important for acrosomal release and post-fertilisation events. Cholesterol release from spermatozoa in vitro resulting in sperm capacitation can be achieved by incubation with a number of substances known to bind cholesterol.

The total amount of cholesterol per spermatozoa may vary more than ten-fold between subjects, but the fertilisation capacity that relates to cholesterol content seem mostly to be correlated to the ability to loose cholesterol during the post-ejaculatory period (Benhoff et al., 1993b). The absolute sterol content of spermatozoa may not be as important as the ratio between free cholesterol and phospholipids. Unexplained infertile men and oligoasthenospermic men may have an increased free cholesterol/phospholipid (C/PL) ratio as compared to normal fertile men but the clinical importance of the cholesterol level appear to be ambiguous (Sugkraroek et al., 1991; Huacuja et al., 1981). Treatment of human spermatozoa with phospholipid preparations increased their binding to the egg zona pellucida concomitant with a reduction in sperm C/PL ratio (Gamzu et al., 1987).

The lipid content of spermatozoa is associated to motility parameters (Connor et al., 1997). Moreover, the C/PL ratio is negatively correlated to motility (Hamamah et al., 1995) and fusogenicity of spermatozoa. Whereas the C/PL ratio may be of importance for the sperm quality, the absolute amount of sterols is still an unresolved matter.

Oocytes

Nothing is presently published concerning the cholesterol content, cholesterol biosynthesis and cholesterol dynamics in mammalian oocytes. This fact is probably due to the low amount of tissue available for analysis.

Statin

A statin is the generic term for a compound that competitively and reversible inhibits the HMG-CoA-reductase enzyme. Applied in vivo, statins mediate a decrease in serum low density lipoproteins (LDL) and an increase in serum high density lipoproteins (HDL) by inhibiting the body's own production of cholesterol. In the present application, the term statin covers any compound, which inhibits de novo cholesterol biosynthesis by inhibiting the hydroxymethylglutaryl coenzyme A reductase (HMG-CoA-reductase) enzyme.

Epidemiologic studies have shown that increased blood cholesterol levels, or, more specifically, increased levels of LDL-cholesterol are causally related to an increased risk of coronary heart disease. There is also substantial evidence that lowering total and LDL-cholesterol levels will reduce the incidence of coronary heart disease. Due to this effect extensive studies over the last decades have generated several compounds capable of inhibiting the HMG-CoA-reductase. These compounds are generally considered safe and widely non-toxic. Only very few reports deal with reproductive hazards of statins. One report on human toxicity excerpts a small but statistically significant reduction in sperm motility in patients with type II hyperlipoprotelnemia receiving oral lovastatin 40 mg daily for 4 months compared with that observed during dietary management alone (Hazardous Substances Data Bank, Sigma Aldrich Norway).

### SUMMARY OF INVENTION

The present invention offers a new and simple method to increase the number and the developmental competence of both female and male germ cells and the organisms resulting from the fertilisation of such germ cells. As a stand-alone method, the fertility improvement is obtained without the side effects of the hormone treatment applied in the most of the presently used methods. Furthermore, the present invention can also be applied in combination with the presently used in vitro methods used in the clinic such as In Vitro Maturation (IVM), and In Vitro Fertilisatlon-Embryo Transfer (IFV-ET) technologies employing varying extents of exogenous gonadotrophin stimulation and germ cell culturing. The temporal sequence of processes relating to in vitro techniques for treatment of sub- or infertility are depicted in FIG. 12. As a supplementary treatment to these conventional methods, the method offers a reduction in the possible side effects.

The present invention relates to methods whereby the number and quality of mammalian gametes and embryos can be increased. The methods applied in vivo or in vitro either inhibit the sterol de novo biosynthesis of germ cells of both sexes and their neighbouring cells or promote sterol efflux from female germ cells prior to fertilisation, or both. The processes that are imagined to be manipulated by the substances described in the present invention are described in FIG. 11. The working mechanism is postulated to involve a lowering of the ratio between free (underivatised) sterol and phospholipid in mammalian germ cells and/or gametes, but other mechanisms that involve the use of mevalonate and post-mevalonate products may likewise be involved.

In one aspect, the invention uses a pharmacological regime that inhibits the endogenous de novo biosynthesis of cholesterol in vivo and/or in vitro by administering or adding a compound that antagonises the 3-hydroxy-3-methylglutaryl coenzyme A reductase enzyme (HMG-CoA-reductase) in germ cells and germ cell supporting tissues. The use of the HMG-CoA-reductase inhibitors, however, are used in a pulsate manner, which contrasts the normal use of these compounds for atheroscleretic prophylactics. Moreover, the HMG-CoA-reductase inhibitors are used in a concentration that may be considerably above the concentration usually used for atheroscleretic prophylactics. The treatment mediates (an) intracellular effect(s) that rescue otherwise non-viable or non-fertilisable germ cells and renders such germ cells viable and prone to fertilisation. In effect, the treatment results in an increased germ cell survival rate and an enhanced germ cell maturation. Also, the treatment leaves the germ cells more prone to maturation and participation in post-maturational events with impact on fertilisation and early embryo development, possibly by affecting the ratio between sterols and phospholipids. The number of germ cells that can be used for later fertilisation is thereby increased, which cause an increasing conception success and an increase in the number of progeny per adult female per reproductive cycle.

The present invention relates to a non-hormonal pharmacological treatment during the follicular phase that results in an increased number of retrieved oocytes and an improvement in their quality in terms of morphology and fertilisability. As a stand-alone technique, the method of the invention operates without the risks of possible side effects of the conventional hormonal treatment. This new non-hormonal pharmacological method, however,is also useful in combination with conventional IVF treatment protocols for women, as well as with applied techniques for livestock or animal breeding in general and under conditions where in vitro techniques are applied as there is a need for improved methods for providing an increased number of retrievable germ cells and improvements in their quality in terms of viability and fertilisability.

### DETAILED DESCRIPTION

In its broadest aspect, the present invention relates to a method for inhibiting the sterol de novo biosynthesis with the effect of decreasing the sterol content of the germ cells and possibly decreasing the ratio between free sterols and phospholipids in at least one mammalian germ cell and/or gamete and thereby increasing the developmental competence of at least one mammalian germ cell, gamete, zygote, early embryo, blastocyst, implanted blastocyst and/or embryo and/or the number of mammalian gametes, zygotes, early embryos, implanted blastocysts, embryos and/or foetus, the method comprising administering a compound which is capable of inhibiting the sterol de novo biosynthesis to a mammal in need thereof.

In another aspect the present invention relates to a method for increasing the sterol efflux from at least one mammalian ovary and/or one mammalian oocyte and/or mamma-lian female gamete and/or ovary derived cell surrounding an oocyte, thereby increasing the developmental competence of said at least one mammalian ovary and/or mammalian oocyte and/or mammalian female gamete and/or ovary derived cell surrounding an oocyte, the method comprising administering a compound or a combination of compounds capable of promoting sterol efflux to a mammal in need thereof.

In the present context the term “germ cell” relates to any mammalian cell capable of producing a gamete and a “gamete” is defined as a mature male or female germ cell possessing a haploid chromosome set and capable of initiating formation of a new diploid individual by fusion with a gamete of the opposite sex, the term thus include e.g. a secondary spermatocyte, spermatide, a spermatozoon, a cumulus enclosed oocyte (oocyte and cumulus cells) and an isolated oocyte.

In the present context the term “mammal” relates to the highest class of the subphylum Vertebrata comprising all animals that nourish their young with milk secreted by mammary glands such as human, horse, cow, rat, mouse, pig, sheep, goat, llama, dog, cat and mink. In a preferred embodiment the present invention relates to humans. In a further preferred embodiment, the present invention relates to mammals that are industrially applicable due to human agriculture and pet industry, such as the animals mentioned above.

The term “zygote” is used in the present context to describe a cell formed by the union of two gametes that develop through pro-nuclei generation and pro-nuclei fusion and initiate the first cellular cleavage, or, in a broader context, as the developing individual produced from gametes. The term “early embryo” is in the present context defined as the two-cell or multicellular organism produced from the zygote until the blastocystic stage, a blastocyst being defined as the stage of the early embryo where a fluid filled cavity, the blastocoel, emerges. The term “implanted blastocyst” relates in the present context to a blastocyst that has initiated hatching from the surrounding eggshell, the zona pellucida, and has just begun the penetration of the uterine lining of the foster mother. An embryo is in the present context defined as the stage of the mammalian development from the termination of the implantatory process and the beginning of the early stages of growth and differentiation that are characterised by cellular cleavage, the laying down of fundamental tissues and the formation of primitive organs and organ systems. Especially this term refers to the developing human individual from the time of implantation to the end of the eighth week after conception, which divides embryo from foetus.

The development from “germ cell” to “foetus” constitutes in the present context a continuum of processes of mammalian development that allow for no intermediate stages to occur that will not be accommodated in one of the aforementioned definitions.

The term “developmental competence” is defined as the ability to develop into a specific unicellular or multicellular stage and includes in the present context any cellular differentiation, development and maturation, fertilisation, as well as post-fertilisation processes covering early embryo development, blastocyst formation, implantation and embryonic and foetal growth.

In a most preferred embodiment of the method of the present invention, the germ cell is a human germ cell such as an oocyte, an immature oocyte retrieved by aspiration of a pre-antral follicle(s), an immature oocyte retrieved by aspiration of small or medium sized antral follicle(s) and/or an immature oocyte obtained after culture of at least one immature follicle at the primordial to the preantral stage, an ejaculated spermatozoa, an immature spermatozoa retrieved from the male reproductive tract, a spermatid or a spermatocyte. The term “immature” relates to germ cells and gametes not capable of conceiving naturally in their present stage.

A compound to be used in the method of the present invention and capable of inhibiting the sterol de novo biosynthesis is preferably selected from the class of compounds that antagonise the 3-hydroxy-3-methylglutaryl coenzyme A reductase enzyme (HMG-CoA-reductase), such as any substance that binds the HMG-CoA-reductase enzyme reversibly or irreversibly, competitively or non-competitively with HMG-COA, and reduces the amount of molar mevalonate produced per molar HMG-CoA-reductase per unit time. One or more compounds may be used, i.e. the compounds may be used separately or two or more compounds may be used in combination.

A HMG-CoA-reductase inhibitor relevant in the present context may be qualified by a test involving the purified solved HMG-CoA-reductase enzyme, freeze-thaw-solved HMG-COA-reductase enzyme, microsomal fractions obtained from gonads or gonadal cells from a mammal. Such cellular and sub-cellular fractions are referred to as “HMG-CoA-reductase enzyme preparations” below.

In an in vitro experiment involving HMG-CoA in a free concentration of 20 μM, a maximum of 10 mg/ml in incubation medium of “HMG-CoA-reductase enzyme preparations”, and incubation at 37° C. in physiological buffer equilibrated in atmospheric air and added the co-factors NADH and NADPH and oxygen regenerating systems, the amount of molar mevalonate produced per molar HMG-CoA-reductase per unit time during steady-state will be:
control value, V_{c}=molar mevalonate produced/mg enzyme preparation/second.

If a substance that inhibits the HMG-CoA-reductase enzyme is added to the same experimental set-up in a concentration that will generate a free concentration of 100 μM of this substance, the free concentration being the concentration of substance diffusing freely and not being bound to macromolecular entities, the amount of molar mevalonate produced per molar HMG-CoA-reductase per unit time during steady-state will be:
inhibitor value, V_{i(100 μM)}=molar mevalonate produced/mg enzyme preparation/second.
If
V_{i(100 μM)≦}0.9×V_{c}
the substance then reduced the amount of molar mevalonate produced per molar HMG-CoA-reductase per unit time to 90% or less of control values, a control value being the conversion rate when no inhibitor is applied. If such condition of at least 10% inhibition at 100 μM substance added is met, the substance qualifies in the present context as an HMG-CoA-reductase inhibitor.

Preferably, said amount is less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, less than 1%. In a preferred embodiment, the compound effectuates an almost complete decrease of the rate of conversion of 3-hydroxy-3-methylglutaryl-coenzyme A into mevalonic acid under the aforementioned conditions.

Other compounds that are capable of decreasing the sterol biosynthesis de novo would be any compound that inhibits any enzymes that are involved in the pre-sterol biosynthetic pathway starting from acetyl-CoA and terminating with lanosterol (FIGS. 2 and 3). Examples of enzymes to be inhibited are mevalonate kinase, phosphomevalonate kinase, pyrophosphomevalonate decarboxylase, isopentenyl pyrophosphate isomerase, dimethylallyl transferase, geranyl transferase, squalene synthase, squalene monoxygenase, oxidosqualene lanosterol cyclase and lanosterol synthase.

The inhibitory potential of such drugs should be assessed by their ability to decrease the amount of enzyme product produced per molar enzyme per unit time in incubated tissue or tissue fragments from gonads and compared to incubations that differ from this by having no potential inhibitor present. The experimental set-up should therefore be similar to those above-mentioned conditions for test of HMG-CoA-reductase inhibitory substances, apart from involving the natural substrate of the enzyme in question instead of HMG-CoA, and this substrate should be used in free concentrations equal to the Km (the Michaelis-Menten constant) of the enzyme-substrate reaction. The inhibitory effect of the substance under conditions similar to the above-mentioned conditions for HMG-CoA-reductase inhibition, i.e. when the substance is applied in concentrations of 100 μM, should be at least 10% in order to be qualified as a sterol synthesis inhibitor.

Such list will include, but not be limited to, zaragozic acid, azasqualene, bisphosphonates (e.g. cycloheptylaminomethylene-1,1-bisphosphonic acid, (3-(1-pyrolidino)-1-hydroxypropylidene-1,1-bisphosphonic acid) and PHPBP (3-(1-piperidino)-1-hydroxypropylidene-1,1-bisphosphonic acid)) and other squalestatins (inhibitors of squalene synthase), imidazoles, pamidronate, alendronate and oxysterols.

In general, the present invention relates to the use of any compound which, during its interaction with the gonadal tissue, will inhibit the de novo sterol biosynthesis and thereby lead to a decreased amount of free sterols in epididymal spermatozoa or oocytes and cumulus-oocyte complexes of mammals possibly resulting ultimately in a lowered sterol/phospholipid ratio in the germ cell supporting cells. The invention relates to any combinations of compounds and administration that through an inhibitory interaction with enzymes involved in the pre-sterol pathway, i.e. the biosynthetic pathway between acetic acid and lanosterol, will lead to decrease of total free sterols in the epididymal spermatozoa or oocytes and cumulus-oocyte complexes of mammals.

The invention relates to any of the above mentioned regimes that result in a decrease of at least 10%, such as at least 5% of free sterols in the epididymal spermatozoa or oocytes and cumulus-oocyte complexes of mammals as compared to a situation where the mammal receives no such treatment. The invention also relates to any combination of a de novo sterol biosynthesis inhibitory regime and known techniques that are applied in order to propagate germ cells in mammals in the need thereof, such as IVM and IVF-ET techniques, stimulation with exogeneous gonadotrophins and/or other techniques.

In the present context the term “gonadotrophin” relates to a mammalian peptide hormone produced by the pituitary or the placenta that contains two peptide subunits, α a and β, of which the α-subunit is shared by them and the β-subunit distinguishes their chemical nature and biological characteristics. Known gonadotrophins so far include follicle stimulating hormone (FSH), luteinizing hormone (LH) and chorion gonadotrophin (CG).

In a presently preferred embodiment of the present invention, the compound used for inhibition of sterol biosynthesis de novo is a statin. Several generic names exist for statins, thus the following list is not intended to limit the scope of the present invention. In a preferred embodiment the statin is selected from the group consisting of atorvastatin, cerivastatin, dalvastatin (RG 12561), fluvastatin, BAY W 62, pravastatin, lovastatin (formerly called mevinolin or monacolin K), 28, HR 780, pravastatin, simvastatin, compatin, methyl-compactin, mevastatin (formerly called campactin or ML-236B), ML-236A, ML-236C, dihydrocompactin, monacolin 3, monacolin L, monacolin M, monacolin X, dihydromonacolin L, mevinolin, 3β-hydroxycompactin acid monosodium salt (pravastatin, CS-514), synvinolin, cerivastatin and nystatin/triamcin.

In the present context a compound capable of promoting sterol efflux (SBTS=Sterol Binding or Transporting Substance) is any substance that during incubation of tissue or cells will lead to an increase in the diffusion of sterol(s) into the medium and therefore efflux of said sterol(s) from the biological material to a sterol acceptor that is initially unsaturated by sterol(s), e.g. serum albumin or another protein, carbohydrate or lipid material.

Sterol efflux can be assayed by incubating conditioned and radio labelled Fu5AH rat hepatoma cells in Eagle's minimum essential medium supplemented with human serum albumin at 3 mg/mL (3% w/v), as well as the putative sterol binding or transporting substance in question. Conditioning and labelling of the cells as well as cell density during culture must conform to literature descriptions in Moya et al. (Moya et al., 1994, Arterioscler. Thromb., 14(7):1056-1065). If Fu5AH rat hepatoma cells are incubated under these conditions for 4 hours at 37° C. and, subsequently, after incubation spent media are separated by centrifugation and washing of the cells in new medium (Eagle's minimum essential medium supplemented with human serum albumin at 3 mg/mL) and radioactivity measured in the two fractions, then:
radioactivity in cells=RA_{cells}
radioactivity in medium=RA_{medium}
where RA_{cells} represents the total radioactivity in the cell fraction and RA_{medium} represents the total radioactivity in the fraction representing medium including the medium from three consecutive washings. If no other substance than serum albumin at 3 mg/mL is present in the medium, then:
radioactivity in cells (standard)=RA_{cells}^{standard}
radioactivity in medium (standard)=RA_{medium}^{standard}

If a SBTS is added, then:
radioactivity in cells (SBTS)=RA_{cells}^{SBTS}
radioactivity in medium (SBTS)=RA_{medium}^{SBTS}

If the substance qualifies as a SBTS in the present context then the fractional release of ³H-Cholesterol from the cells is increased 50% or more after 4 hours of incubation at 37° C., i.e.:
(RA_{medium}^{SBTS})/(RA_{cells}^{SBTS})≧150%×(RA_{medium}^{standard})/(RA_{cells}^{stsndard})

In a preferred embodiment, the amount of SBTS added to the medium results in an increase in the medium osmolarity of 0.001-10%. In another preferred embodiment the amount of SBTS added to the medium is in the range of 0.001-10 mM. In yet another preferred embodiment the amount of SBTS added to the medium is in the range of 0.01-50%o (w/w), such as but not limited to 0.5-4%o.

Interesting compounds capable of promoting the sterol efflux could be cyclodextrins, chemically modified cyclodextrins, such as but not limited to sulphated cyclodextrins, high density lipoproteins (HDL), an apoprotein derived from HDL, sterol carrier protein I and II or any other protein capable of solubilizing sterols.

In one embodiment the compound or a combination of compounds capable of promoting the sterol efflux is selected from the group consisting of cyclodextrin, chemically modified cyclodextrins, such as but not limited to sulphated cyclodextrins, high density lipoproteins (HDL), an apoprotein derived from HDL, and sterol carrier protein I and II.

In a most preferred embodiment of the present invention the compound capable of promoting the sterol efflux is a cyclodextrin.

In the present context the term “cyclodextrin” relates to a homologous group of cyclic glucans consisting of alpha-1,4 bound glucose units obtained by the action of cyclodextrin glucanotransferase on starch or similar substrates. Cyclodextrins form inclusion complexes with a wide variety of substances and thereby co-solubilize substances that are otherwise insoluble or low-soluble in water or buffers. Cyclodextrins differ from one another by the number of glucopyranose units in the structure. The parent cyclodextrins contain 6, 7 and 8 glucopyranose units and are referred to as alpha (a-), beta (β-), and gamma (g-) cyclodextrin respectively. As an example, FIG. 13 shows the generic structure of sulphated-cyclodextrin (R═H). If R═CH2CH(OH)CH3 the structure represents hydroxypropyl-β-cyclodextrin used in example 8 below.

The amount of statin or compound capable of inhibiting sterol biosynthesis de novo that should be administered to the mammal in the need thereof depends on the particular statin and/or compound chosen and is between 0.01-100 mg per kg body weight per day, such as 0.05-90 mg per kg body weight per day, 0.1-80 mg per kg body weight per day, 1-50 mg per kg body weight per day, 1-25 mg per kg body weight per day, 1-10 mg per kg body weight per day, 1.0-10 mg per kg body weight per day, 2.0-10 mg per kg body weight per day, 2.5-10 mg per kg body weight per day, 3.0-10 mg per kg body weight per day, 4.0-10 mg per kg body weight per day, 5.0-10 mg per kg body weight per day, 6.0-10 mg per kg body weight per day, 7.0-10 mg per kg body weight per day, 8.0-10 mg per kg body weight per day, 9.0-10 mg per kg body weight per day or between 1.0-9.0 mg per kg body weight per day, 2.0-8.0 mg per kg body weight per day, 2.5-7.5 mg per kg body weight per day, 4.0-6.0 mg per kg body weight per day or 4.5-5.5 mg per kg body weight per day.

The amount of compound capable of promoting sterol efflux that should be administered to the mammal in the need thereof depends on the particular compound chosen but will likely be between 0. 01-1000 mg per kg body weight per day, such as 0.5-900 mg per kg body weight per day, 1-800 mg per kg body weight per day, 10-500 mg per kg body weight per day, 10-250 mg per kg body weight per day, 10-100 mg per kg body weight per day, 20-100 mg per kg body weight per day, 25-100 mg per kg body weight per day, 30-100 mg per kg body weight per day, 40-100 mg per kg body weight per day, 50-100 mg per kg body weight per day, 600-100 mg per kg body weight per day, 70-100 mg per kg body weight per day, 80-100 mg per kg body weight per day, 90-100 mg per kg body weight per day or between 10-90 mg per kg body weight per day, 20-80 mg per kg body weight per day, 25-75 mg per kg body weight per day, 40-60 mg per kg body weight per day or 45-55 mg per kg body weight per day.

The reproductive state of the female mammal is cyclic with a complex interaction between the hypothalamus, anterior pituitary and the ovaries leading to the process of ovulation. In humans the cycle is repeated with an average period around 28 days (range 21-35 days). The first phase, menstruation, lasts 3-5 days. The first day of a cycle in a human is the first day of the first phase, that is the first day of menstrual bleeding. The second follicular phase of the human ovary corresponds to the proliferative phase of the endometrium and is least 5-16 days (i.e. highly variable). Then follows an ovulatory phase (36 hours) and finally a luteal phase, which corresponds to the secretory phase of the endometrium and is usually more constant at around 14 days. In the present context, the moment of ovulation is the timing of the discharge of an ovum from the ovary.

The period for which it is necessary to use treatment regimen of the present invention is calculated within each ovulatory cycle. The ovulatory cycle differs between the individual species among the mammals, therefore the optimum period where the administration is needed should preferably be calculated in regards to each individual species based on their ovulatory cycle. In several mammals, the duration of the ovulatory cycle differs highly from the human cycle. The average duration of the ovulatory cycle in various mammals are given in table 1 below:

TABLE 1

| | Mammal | | | | | | | | | | | | |
|---|---|---|---|---|---|---|---|---|---|---|---|---|---|
| | Man | Horse | Cattle | Goat | Swine | Sheep | Hamster | Mouse | Rat | Rabbit | Mink | Dog | Cat |
| average | 28 | 21 | 21 | 20 | 21 | 16 | 4 | 5 | 5 | 16 | 8 | 15 | 18 |
| length | | | | | | | | | | | | | |
| (days) | | | | | | | | | | | | | |

According to the method of the present invention, a female mammal in need thereof is to be administered a compound which is capable of inhibiting the sterol de novo biosynthesis and/or promoting sterol efflux during a period of 0 hour-12 days before ovulation, such as 0 hour-11 days, 0 hour-10 days, 0 hour-9 days, 0 hour-8 days, 0 hour-7 days, 0 hour-6 days, 0 hour-5 days, 0 hour-4 days, 0 hour-3 days, 0 hour-2 days or 0 hour-24 hours before ovulation.

The administration of a compound which is capable of inhibiting the sterol de novo biosynthesis and/or promoting sterol efflux could be extended up til 3 days after ovulation, such as 1 hour, 2 hours, 5 hours, 10 hours, 15 hours, 20 hours, 24 hours, 36 hours, 48 hours, 60 hours or 72 hours after ovulation.

One important aspect of the present invention relates to a pharmaceutical composition comprising a combination of a compound capable of inhibiting the sterol de novo biosynthesis and a compound capable of increasing the sterol efflux, optinonally in a combination with a pharmaceutically acceptable carrier.

In one specially preferred embodiment of the present invention a synergistic effect between the statin and the cyclodextrin is obtained due to the temporal coincidence of the two different processes that have an effect on the sterol content of the cell thereby creating an effect that is above the summation of the two regimes used separately, preferably by at least a factor 3.

A preferred composition according to the invention is the combination comprising a statin and a cyclodextrin. In a further embodiment the invention relates to the use of a statin and a cyclodextrin together with a pharmaceutically acceptable carrier for the preparation of a medicament for increasing the fertility of a mammal in the need thereof.

Growth factors have long been used in the clinic, thus in a preferred embodiment the present invention relates to compositions containing a statin and a cyclodextrin further comprising a growth factor. Such growth factors could be gonadotropins (FSH, LH, CG), insulin-like growth factors (IGFs), epidermis growth factors (EGFs), Insulin, growth hormone, interleukines or other peptide hormones.

In a preferred embodiment the growth factors is selected from the group consisting of gonadotropins (FSH, LH, CG), insulin-like growth factors (IGFs), epidermis growth factors (EGFs), Insulin, growth hormone, interleukines and other peptide hormones.

Another important aspect of the present invention relates to a pharmaceutical composition comprising a combination of a compound capable of inhibiting the sterol de novo biosynthesis and a growth factor. In a preferred embodiment said compound is a statin.

Yet another important aspect of the present invention relates to a pharmaceutical composition comprising a combination of a compound capable of promoting the sterol efflux and a growth factor. In a preferred embodiment said compound is a cyclodextrin.

In a preferred embodiment of the present invention the compound and/or any of the compositions mentioned above are administrated to the mammal 1-10 times a day, such as 1 time a day, 2 times a day, 3 times a day, 4 times a day, 5 times a day, 6 times a day, 7 times a day, 8 times a day, 9 times a day or 10 times a day.

The compound and/or compositions may also be administered one to several times or on a continuous basis via a tissue-pump device during this period. The compound and/or compositions may be administered one to several times on a continuous basis via a tissue-pump device during this period. The compound(s) and/or compositions may be administered in a formulation that retards its release in the body. Such formulation will provide for the possible administration of the compound(s) and/or compositions in a period of say 0-30 days prior to the aforementioned period, but effecting a tissue exposition in the very same period by a slow release in vivo. Such administration will thereby provide the similar desired effect on the germ cell development as an administrative regime resulting in an immediate body release.

The present invention also relates to the use of any of the above mentioned compositions for the preparation of a pharmaceutical composition for use in any of the methods of the invention.

It is known to a person skilled in the art that an egg is an ovum that has undergone chromosomal reduction, and therefore may be ready for fertilisation, and takes the form of a relatively large non-motile gamete which contributes to most of the cytoplasm of the zygote. These eggs, even though released as mature and ready to be fertilised, somehow could lack the ability to complete the fertilisation process, which is helped by adding a compound capable of inhibiting the sterol de novo biosynthesis.

In the examples, it is demonstrated that Mevacor®, a pharmaceutical product of the statin group that works by inhibiting HMG-CoA-reductase, the key regulatory step of cholesterol biosynthesis de novo, causes a decrease in serum cholesterol, and, as shown here, influences the amount of free cholesterol in organs like the liver and the ovary (example 3). In parallel to this, the ovaries respond by producing a larger number of fertilisable oocytes and/or increase the developmental competence of the prevalent ovulated eggs, which is evidenced by the increased number of 2-cells in tuba following mating with normal (untreated) male mice (example 1).

The improvement of statins with respect to meiotic maturation is demonstrated by example 4, which shows that applying a statin (Compactin, Sigma) to the culture medium mediates oocyte meiotic maturation in vivo. Moreover, the effect appears to be additive to the well-known meiosis maturation effect of gonadotrophins (here FSH).

Metabolic data are included that show correlation between oocyte maturation and de novo biosynthesis of cholesterol. The replacement of Mevacor® with Compactin in vitro was necessary because the active component in Mevacor®, lovastatin, is only active after bioconversion in the liver. The cholesterol-lowering enhancement by statins in comparison to hCG as the more natural ovulatory trigger is evidenced by example 3. The overall decrease of free cholesterol during hormone induced oocyte development and ovulation in the mouse is evidenced by data represented in example 5.

The improvement provided by a statin with respect to fertilisation is demonstrated by example 7, which shows that applying a statin (Compactin, Sigma) to the IVM culture medium mediates improved oocyte fertilisation by sperm using IVF. It is demonstrated that the presence of compactin during IVM mediated an increased number of fertilised oocytes after IVF as compared to a control incubation and an incubation with FSH. The rate of generated oocytes remains the same (FIG. 8). It is surprisingly that the statin has an enhanced effect as compared to the addition of FSH. In FIG. 9 the results show that cells treated with Mevinolin has reached a more progressed state after 72 hours of culture following IVF, as compared to the two aforementioned groups for comparison. The rate of degeneration after 72 hours of culture is lower in the Mevinolin treated group as compared to the two aforementioned groups for comparison, which may correspond to the fact that the oocytes cultured in the presence of Mevinolin displayed a high degree of cumulus expansion after IVM which was not visible during the microscope examination. Cumulus expansion is pre-requisite for proper oocyte maturation and fertilisation preparation.

The improvement of a sterol binding or transporting substance with respect to fertilisation, exemplified by hydroxypropyl-β-cyclodextrin, is demonstrated by example 8, which shows that applying hydroxypropyl-β-cyclodextrin to the IVM culture medium mediates improved oocyte fertilisation by sperm using IVF, as shown in FIG. 10. The rate of oocyte degeneration was not affected by the present range of cyclodextrins.

A background for the present invention is the prior knowledge that loss of free sterol from the plasma membrane is a pre-requisite for proper terminal maturation of the mammalian spermatozoa. However, the present invention is based on three new major ideas.

1) the membrane sterol decrease is a biochemical process that also takes place during final pre-fertilisation maturation of the oocyte. No knowledge on ovarian or oocyte loss of sterol prior to fertilisation has hitherto been presented in the scientific literature or elsewhere.

2) The sterol loss and/or processes related to a lowered intracellular sterol content can be enhanced in germ cells of both sexes by applying inhibitors of the de novo sterol biosynthesis pathway. The inhibition will not lead to full sterol compensation by the germ cell or germ cell environment by for instance increasing lipoprotein intake or increasing sterolester hydrolysis. This is probably because the vast majority of the sterols of the germ cell environment closely prior to fertilisation are produced in situ under natural conditions. The sterol biosynthesis inhibitory action will lead to a significant decrease in the membrane sterol content that may substitute the natural sterol loss stimulated by hormones. Moreover, the natural sterol loss will be enhanced by the inhibition of the sterol biosynthesis de novo.

3) The de novo sterol biosynthesis inhibition mediates important maturatlonal processes per se, e.g. meiotic maturation, and constitutes a mechanism that mediates pivotal processes for germ cells development apart from capacitation in spermatozoa. Also, the membrane sterol loss mediates important maturational processes.

The present application provides experiments wherein statin is used in order to manipulate the level of free sterol negatively in the oocyte and the oocyte environment. The experiments relating to the present invention show that enhancement of the natural sterol loss during final oocyte maturation results in increased viability and fertilisability of oocytes, probably by rescuing a number of “borderline” oocytes that would otherwise have become atretic and thereby lost during the hormone dependent growth phase. Moreover, it is contemplated that giving statin to a male mouse in vivo results in a decreased sterol de novo biosynthesis and/or decrease of phospholipid/sterol ratio in the motile cells (normal spermatozoa) of the epididymis.

The present invention demonstrates (FIG. 5) that hormone stimulated and statin treated prepubertal female mice produce on average about 70% more 2-cells as compared to the same mice stimulated with hormone alone. It is contemplated that even higher percentages may be obtained by tuning the statin dose and period of treatment. The example demonstrates that applying a statin to a female mammal increases the developmental competence of its gametes.

The present invention demonstrates that a statin can be administered during the follicular phase maturation of follicles in vivo with the effect on enhancing the maturational events. The effect possibly relies on a decrease in the amount of total free sterols in the germ cell as compared to animals where de novo sterol biosynthesis is taking place, which leads to a decreasing sterol to phospholipid ratio in the plasma membrane resulting in a germ cell more prone to participation in the process of fertilisation. Also, in vitro oocyte maturation and fertilisatlon will benefit from a treatment that inhibits sterol de novo biosynthesis, resulting in increased rates of meiosis and enhanced post-meiotic maturation.

The well-known process of sterol loss during sperm capacitation in vivo or in vitro has not been linked to the intracellular de novo sterol production. Cholesterol is present in cells in the internal and external membranes, and a steady loss to the extracellular space is taking place in all cells depending on the nature of the extracellular milieu. Moreover, cholesterol is used as a substrate for synthesis of e.g. bile acids and steroids. On the other side, free cholesterol is obtained by cells from internal cholesteryl-ester stores, external lipoprotein sources and de novo synthesis. Inhibition of one of these processes generally lead to a potentiation of one of the other processes in order to obtain a constant level of sterols in the cell membranes and organelles.

These processes altogether ensure a cholesterol steady state in the cells, and it is not imaginable that the inhibition of a single of the cholesterol delivering processes will change the overall picture of cholesterol homeostasis and processes relying on cholesterol usage. For instance, the production of steroids in the female gonad takes place without being affected by a complete blockage of the sterol de novo production, as exemplified by example 3 in the present application. A blocking of the contribution to the steady state from the sterol de novo synthesis pathway could therefore likewise be expected to increase cholesterol intake (lipoprotein intake and release of free cholesterol) and intracellular mobilisation (cholesteryl-ester hydrolysis). However, the present examples 3 and 6 shows that a decrease per weigh unit or phospholipid unit is the result in the ovary and in the motile cellular fraction in the epididymis, the storing site of mature sperm in the male. Example 1 and 9 shows that this sterol decrease is accompanied by an improved germ cell development and/or quality.

On the other hand, if it was imaginable that a blockage of the de novo sterol biosynthesis would lead to a decreased usage of sterols for other biochemical processes resulting in a decreased output of e.g. steroids, this would severely diminish the potential use of the present method in pro-fertility treatment by interfering with steroid output and germ cell maturation processes relying on steroids. The present method demonstrates the surprising finding that a pharmacological suppression of the de novo sterol biosynthesis leads to an increased germ cell quality in the female. The regime does not affect steroidogenesis, i.e. processes that rely on cholesterol usage, negatively. A sterol usage dichotomy is therefore operating in the female germ cell and/or germ cell environment that enables the cells to respond by decreasing the sterol output in one cellular and/or biochemical compartment and at the same time leaving another compartment unaffected with respect to sterol usage and/or presence.

The present invention relates to the use of known generic compound for improving the fertility of a mammal by inhibiting sterologenesis de novo. The underlying mechanism in the demonstrated fertility improvement may be a modulation of the sterol/phospholipid ratio. The present invention shows that decreasing the sterol biosynthesis de novo in mammals by pharmacological intervention produces female germ cells with an improved developmental competence. The cross-sexual aspects of the sterol decreasing mechanism has guided the belief that also male germ cells will benefit from a regime built on sterol inhibition prior to fertilisation.

It is known to the person skilled in the art that the timing of intercourse causing pregnancy vary between the different species. Therefore the present invention relates to situations where mammals have difficulties in producing offspring, such as in situations where the female mammal is unable to achieve a pregnancy after more than one day of unprotected intercourse, such as 2 days, 3 days, 4 days, 5 days, 10 days, 15 days, 20 days, 30 days, 1 month, 2 months, 3 months, 4 months, 5 months, 6 months, 7 months, 8 months, 9 months, 10 months, 11 months, 1 year, 2 years, 3 years, 4 years, 5 years, 10 years, 15, years, 20 years or more than 60 years of unprotected intercourse.

It is known that the timing of unprotected intercourse described above is related to the period in which the mammals regularly have intercourse with the purpose of conceiving.

Since the present application discloses the new and surprising observation that inhibition of sterol biosynthesis de novo improves maturation of mammalian germ cells, the present invention also relates to situations in which the mammal does not suffer from infertility, but merely wishes to obtain a higher conception chance and thereby faster conception, especially in respect to female humans in the age range from 28 to 42 in which the conception level decreases proportionally with age. The present invention also relates to human females that may benefit from the treatment by obtaining twin-carriage or tri-carriage. In general, the present method relates to livestock and other economically valuable mammals that could benefit from it by producing an increased number of foetuses per pregnancy.

The focus of infertility is of course not only based on the female. In the present invention, the effect on the germ cell maturation in the female is hypothesised to be reflected in the male germ cells. Thus, in another embodiment the present invention relates to a method for inhibiting the sterol de novo biosynthesis in at least one mammalian germ cell and/or gamete and thereby increasing the developmental competence of at least one mammalian gamete, zygote, early embryo, implanted blastocyst and/or embryo and/or the number of mammalian gametes, zygotes, early embryos, implanted blastocysts and/or embryos, the method comprising administering to a male a compound which is capable of inhibiting the sterol de novo biosynthesis during a period of 0 hour-12 days before ejaculation, such as 0 hour-11 days, 0 hour-10 days, 0 hour-9 days, 0 hour-8 days, 0 hour-7 days, 0 hour-6 days, 0 hour-5 days, 0 hour-4 days, 0 hour-3 days, 0 hour-2 days and 0 hour-24 hours before ejaculation. The term ejaculation relates in the present context to any discharging of semen from the male spermatic duct.

One important aspect of the present invention relates to a method for improving the number and developmental competence of at least one mammalian germ cell and/or gamete in vitro and thereby increasing the fertilising ability of at least one mammalian gamete and/or the developmental competence of the resulting zygote, early embryo, implanted blastocyst and/or embryo and/or the number of mammalian gametes, zygotes, early embryos, implanted blastocysts and/or embryos, the method comprises adding to a medium a substance which is capable inhibiting the sterol de novo biosynthesis in cells.

Another aspect of the present invention relates to a method for increasing the sterol efflux from at least one mammalian ovary and/or mammalian oocyte and/or mammalian female gamete and/or ovary derived cell surrounding an oocyte, thereby increasing the developmental competence of said at least one mammalian ovary and/or mammalian oocyte and/or mammalian female gamete and/or ovary derived cell surrounding an oocyte, the method comprising adding to a medium a compound or a combination of compounds capable of promoting the sterol efflux in any of the above mentioned cells or tissues.

It is an object of preferred embodiments of the present invention to provide a variation of cell culture media comprising the various compounds capable of decreasing the phospholipid/sterol ratio in a cell either by increasing the sternol efflux and/or inhibiting the sterol de novo biosynthesis. In one preferred embodiment the present invention thus relates to a cell culture medium comprising a cyclodextrin. In another preferred embodiment the present invention relates to a cell culture medium comprising a statin. In a most preferred embodiment the present invention relates to a cell culture medium comprising a statin and a cyclodextrin.

Several commercially available media exist which support the respiration and/or maturation of gametes and are also capable of culturing mammalian zygotes, early embryos, implanted blastocysts and/or embryos such as, but not limited to, Ham's F10, Menezo B2, Medi-Cult Universal IVF medium, Dulbecco's modified eagle medium (DMEM), McCoy's 5A, Waymouth's medium, TCM-199, MEM, RPMI-1640 and Leibovitz L-15.

In the present context an in vitro medium is defined as a medium which supports the respiration and/or maturation of at least one oocyte in its somatic cellular context or alone and/or at least one spermatozoo, said medium may comprise cell culture media which support in vitro fertilisation (IVF) protocols and/or support survival and maturation of gametes in vitro. Several parameters for a medium that is likely to be useful for in vitro fertilisation (IVF) protocols and/or support survival and maturation of gametes can be established to define said medium. Foremost the concern to pH, osmolarity and the content of the buffered air under which the media is cultured is of high importance. Thus, in the present context an in vitro medium has preferably an osmolarity between 240 to 320 mOsm/l, a pH of 7 to 7.8, and is CO₂-buffered under 3-7% CO₂ in atmospheric air or 53-7% CO₂, 1-5% O₂ and 88-96% N₂.

An osmolarity between 200 to 370 mOsm/l may more specifically relate to levels such as 250 to 310 mOsm/l, 260 to 300 mOsm/l, 270 to 290 mOsm/l, 275 to 285 mOsm/l, 276 to 284 mOsm/l, 277 to 283 mOsm/l, 278 to 282 mOsm/l or 279 to 281 mOsm/l. In a presently preferred embodiment, the osmolarity is about 280 mOsm/l.

In this context a pH between 7 to 7.8 pH may more specifically relate to levels such as 7.1 to 7.7, 7.2 to 7.6, 7.3 to 7.5, 7.35 to 7.45, 7.36 to 7.44, 7.37 to 7.43, 7.38 to 7.42 e.g. 7.39 to 7.41 pH. In a preferred embodiment, the pH is about 7.4.

Without the intention to limit the scope of the present invention, further general characteristics for an IVF-medium could be that the medium contains 111 to 171 mEq/l Na⁺, 2.1 to 6.1 mEq/l K⁺, 96 to 146 mEq/l Cl⁻, 22.6 to 26.6 mEq/l HCO₃⁻, 0.3 to 1.2 mM Ca⁺⁺, 29 to 69 mg/l phosphorus, 0.56 to 1.56 g/l glucose, 0-50000 IU/l penicillin, 0-50000 IU/l streptomycin, 0.1 to 10.0 g/l albumin, 0-75 IU/l FSH, 0-250 IU/l LH and 0-6 g/l of total protein. Other possible additives include foetal calf serum, human serum such as maternal serum, antioxidants such as tocopherol and/or ascorbic acid, hormones, Mg⁺⁺, pyruvate, lactate, phenol red, purines, pyrimidines, amino acids and cholesterol. Generally, the medium is to be cultured at 37° C. (±1° C.).

In a preferred embodiment of the present invention the cell culture medium relates to a chemically defined cell culture medium. The term “chemically defined medium” is used to denote a medium without biologically extracted serum substances, and where all components and their concentrations are known and described. If hormones or serum derived substances are to be added to the medium, recombinant hormones or serum derived substances are preferred. In an alternative embodiment, the culture medium contains BSA or HSA obtained by recombinant methods, thereby eliminating the inter-mammal serum contact. In a much preferred embodiment the immature human gametes are cultured in a chemically defined medium without addition of directly serum-derived products or the patients' own serum or any other serum product derived directly from a mammal, such as a human or cattle.

Growth factors such as, but not limited to, gonadotropins, are presently used in IVM treatment. Thus a preferred embodiment of any of the media described above further relates to a cell culture medium further comprising at least one growth factor, wherein said growth factor is selected from the group comprising gonadotropin (FSH, LH, CG), IGF, EGF, Insulin, growth hormone, interleukines or other peptides.

The method of the present invention will preferably start with immature or not fully matured female gamete. In a woman an immature oocyte will be recognised as an oocyte with tight cumulus masses, no polar bodies or germinal vesicles visible. These oocytes are readily recognised by a person involved in routine IVF-treatments as being immature oocytes, and thus one embodiment of the present invention relates to a method for maturating an immature oocyte obtained from a stage between the primordial follicle and the pre-antral stage comprising culturing said immature oocyte in a cell culture medium as described above.

In a preferred embodiment, the amount of statin administered to an in vitro medium depends on the particular statin and is 0.01-1000 μM, 0.05-1000 μM, 0.1-1000 μM, 0.5-1000 μM, 1-1000 μM, 5-1000 μM, 10-1000 μM, 20-1000 μM, 25-1000 μM, 30-1000 μM, 50-1000 μM, 75-1000 μM, 100-1000 μM, 200-1000 μM, 300-1000 μM, 400-1000 μM, 500-1000 μM, 750-1000 μM or between 10-900 μM, 25-750 μM, 50-500 μM, 100-400 μM, 200-300 μM, 0.01-20 μM, 0.01-15 μM, 0.05-15 μM, 0.1-10 μM or 1-10 μM.

The culturing of at least one oocyte in an in vitro medium according to the method of the present invention is optimised to inhibit the sterol de novo biosynthesis and/or promoting sterol efflux and/or decreasing the ratio between free sterol and phospholipid by culturing the at least one oocyte in its somatic cellular context or alone in an in vitro medium between 1 second and 15 days such as between 1 second and 14 days, between 1 second and 13 days, between 1 second and 12 days, between 1 second and 10 days, between 1 second and 9 days, between 1 second and 8 days, between 1 second and 7 days, between 1 second and 6 days, between 1 second and 5 days, between 1 second and 4 days, between 1 second and 3 days, between 1 second and 2 days, between 1 second and 1 day, between 1 second and 23 hours, between 1 second and 22 hours, between 1 second and 21 hours, between 1 second and 21 hours, between 1 second and 20 hours, between 1 second and 19 hours, between 1 second and 18 hours, between 1 second and 15 hours, between 1 second and 12 hours, between 1 second and 11 hours, between 1 second and 10 hours, e.g. between 1 second and 5 hours.

The culturing of spermatozoa in an in vitro medium by the method according to the present invention is optimised to inhibit the sterol de novo biosynthesis and/or promoting sterol efflux by culturing the spermatozoa in a in vitro medium between 1 second and 8 days such as between 1 second and 7 days, between 1 second and 6 days, between 1 second and 6 days, between 1 second and 4 days, between 1 second and 3 days, between 1 second and 2 days, between 1 second and 1 days, between 1 second and 23 hours, between 1 second and 22 hours, between 1 second and 21 hours, between 1 second and 21 hours, between 1 second and 20 hours, between 1 second and 19 hours, between 1 second and 18 hours, between 1 second and 15 hours, between 1 second and 12 hours, between 1 second and 11 hours, between 1 second and 10 hours, e.g. between 1 second and 5 hours.

IVF treatments generally involve hormone treatment of the female mammal in the need thereof. Even though hormone treatment does have several side effects, it is still one of the most effective treatments of infertility today. A standard IVF treatment of an infertile couple usually involves a sample of mature egg collected from the woman after treatment with exogeneous hormones. The regime usually implies that the woman is treated with gonadotrophin releasing hormone agonists or antagonists, which both result in a substantially decreased extent of gonadotrophin release from the pituitary. Exogeneous gonadotrophins are subsequently provided by intramuscular or intraperitoneal injections, which secure that a number of oocytes will grow and mature. The rationale behind such treatment is that the exact amount of circulating gonadotrophins in the woman can be deliberately controlled, with the result that the number of oocytes that subsequently can be used for fertilisation is optimised and in any case will exceed the number of oocytes—usually only a single—that is ready for fertilisation in an untreated (spontaneous cycle) woman. In usual protocols this treatment involves that oocytes are retrieved from the woman by aspiration prior to the time when the administered gonadotrophins would have triggered the release of the ovum/ova from the ovary into the tuba.

The additive and possibly synergistic effect of administrating a compound capable of inhibiting the sterol de novo biosynthesis and/or a compound capable of promoting sterol efflux in combination with an administration of gonadotrophins or other hormone(s) known to the person skilled in the art to be used for IVF treatment is a preferred embodiment of the present invention.

In the female mammal, the combination of treatment in vivo by a compound capable of inhibiting the de novo sterol biosynthesis and/or a compound capable of promoting sterol efflux with the treatment in vivo of exogeneous gonadotrophins, a gonadotrophin-releasing hormone (gnrh) or a gnrh agonist and/or antagonist, provides a additive/synergistic effect that increases the number of mature ova ready for fertilisation in vivo or in vitro and/or the number of fertilised ova per total number of ova and/or the number of blastocysts per fertilised ova, and/or the number of implanted blastocysts per developed blastocyst and/or the number of delivered offspring per reproductive cycle as compared to a standard IVF treatment.

Also, the administration of de novo sterol biosynthesis inhibitors and/or compounds capable of promoting sterol efflux to women during the hormone stimulation pre-aspiration period will possibly lead to the saving of exogeneous gonadotrophins to be used for obtaining a certain number of mature oocytes, fertilised ova, blastocysts, implants and/or subsequent pregnancy. Such hormone saving regime will represent a side-effect reducing and cost-reducing alternative to the use of exogeneous hormones alone, and this constitutes yet another important scope of the present invention. Amongst the unwanted side effects are the development of the ovarian hyperstimulatory syndrome (OHSS) and the polycystic ovarian syndrome (PCO). The use of statins as an adjuvant during the stimulation of the ovaries in vivo will result in a reduction in the gonadotrophin dose used in vivo and a subsequent reduction in the incidence of these side effects.

In the present context the term “gonadotrophin-releasing hormone” or “gnrh” relates to an endogenous mammalian peptide hormone produced by the hypothalamus that stimulates the pituitary or other organs or tissues to release gonadotrophins. Other designations of the hormone class include luteinizing hormone-releasing factor and luteinizing hormone-releasing hormone.

In the present context the term “gonadotrophin-releasing hormone agonist” relates to any substance that will stimulate the mammalian gonadotrophin-releasing hormone receptor in the pituitary or elsewhere to produce second messengers normally involved in the signal-transduction between binding of the gnrh-receptor and gnrh and gonadotrophin-production and/or gonadotrophin-release from the pituitary or elsewhere.

In the present context the term “gonadotrophin-releasing hormone antagonist” relates to any substance that will inhibit gnrh to bind gnrh-receptors and/or inhibit the signal-transduction normally involved in gnrh-binding, thereby hindering the cell bearing the gnrh-receptor to produce and/or release gonadotrophins upon stimulation with gnrh.

Standard IVF treatment involves that the male deliver a semen sample obtained by masturbation or electro-stimulated ejaculation. The synergistic effect of administrating a compound capable of inhibiting the sterol de novo biosynthesis and/or a compound capable of promoting sterol efflux prior to obtaining the semen sample to be used for IVF treatment is likewise a preferred embodiment of the present invention. In the male mammal, the combination of treatment in vivo by a compound capable of inhibiting the de novo sterol biosynthesis and/or a compound capable of promoting sterol efflux provides a additive synergistic effect that increases the number of mature spermatozoa ready for fertilisation in vivo or in vitro and/or the number of fertillsed ova per total number of ova and/or the number of blastocysts per fertilised ova, and/or the number of implanted blastocysts per developed blastocyst and/or the number of delivered offspring per reproductive cycle as compared to a standard IVF treatment.

The concept of IVF treatment also involves that the germ cells after retrieval from the male and female are cultured under appropriate conditions where a number of spermatozoa are mixed with at least one oocyte. Yet another preferred embodiment of the present invention is the use of de novo sterol biosynthesis inhibitors and/or compounds capable of promoting sterol efflux added to the medium in which the ova and/or spermatozoa are incubated prior to mixture as well as to the medium in which the spermatozoa and ova are incubated in order to obtain a fertilised ovum. Such addition will result in an increased number of mature ova ready for fertilisation in vivo or in vitro and/or the number of fertilised ova per total number of ova and/or the number of blastocysts per fertilised ova, and/or the number of implanted blastocysts per developed blastocyst and/or the number of delivered offspring per reproductive cycle as compared to a standard IVF treatment.

It should be understood that any feature and/or aspect discussed above in connection with the methods according to the invention apply by analogy to the uses according to the invention.

### LEGENDS TO FIGURES

FIG. 1

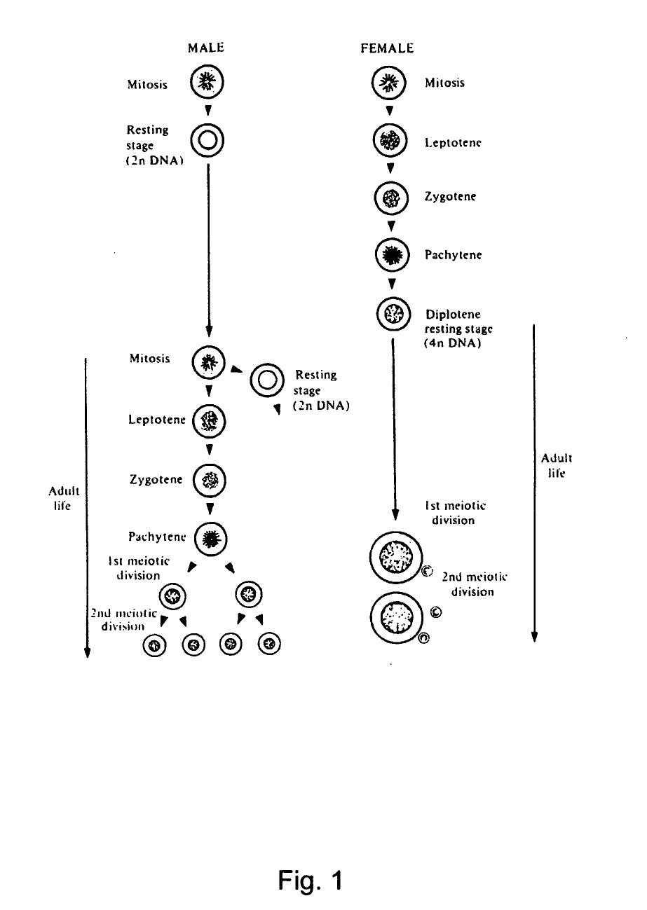

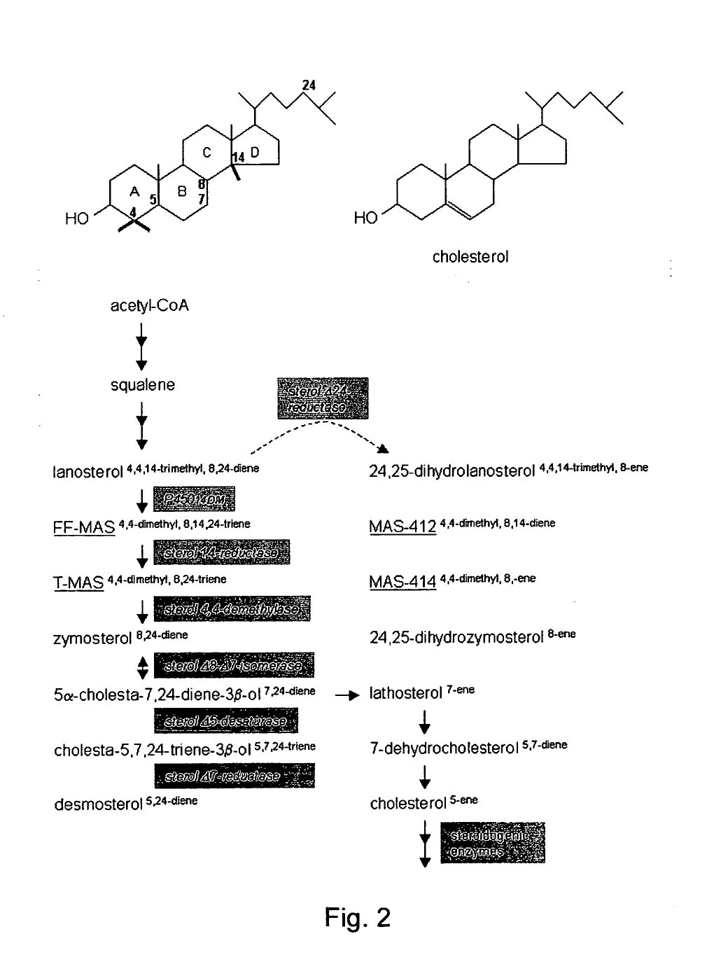

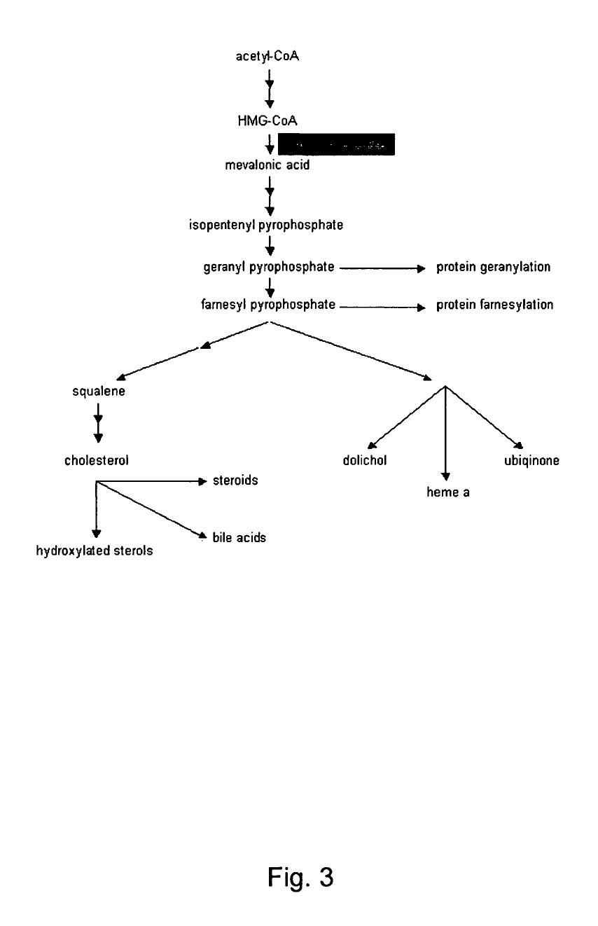

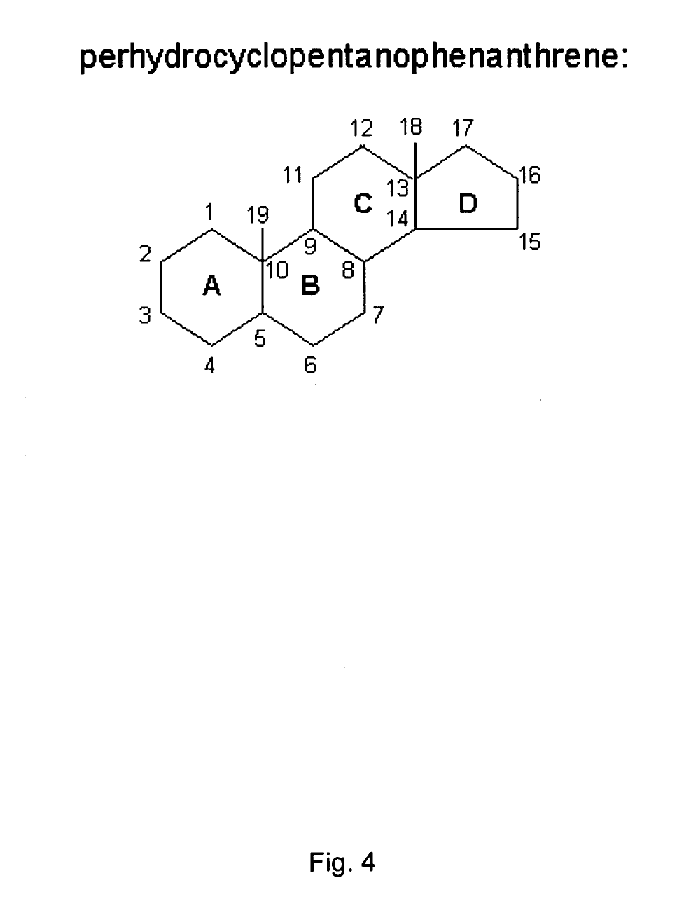

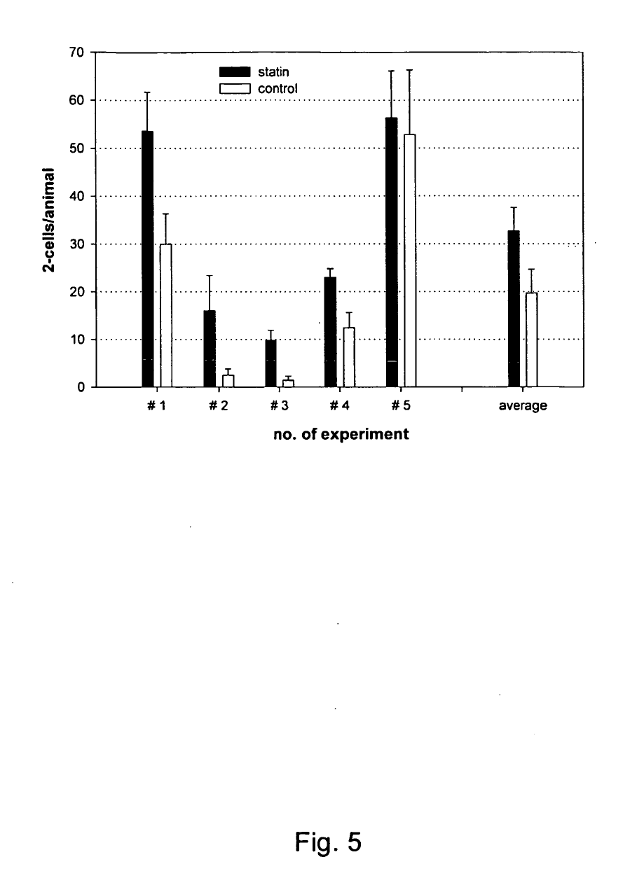

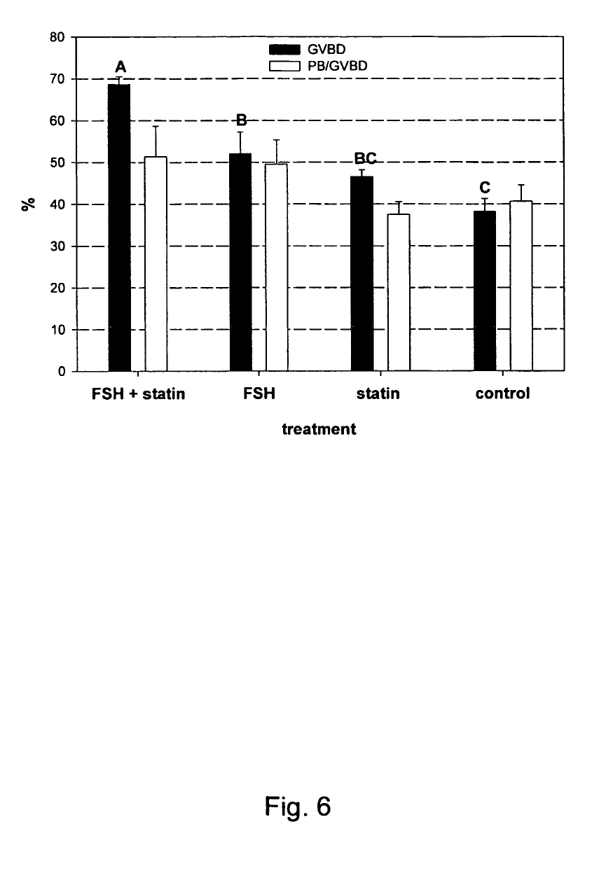

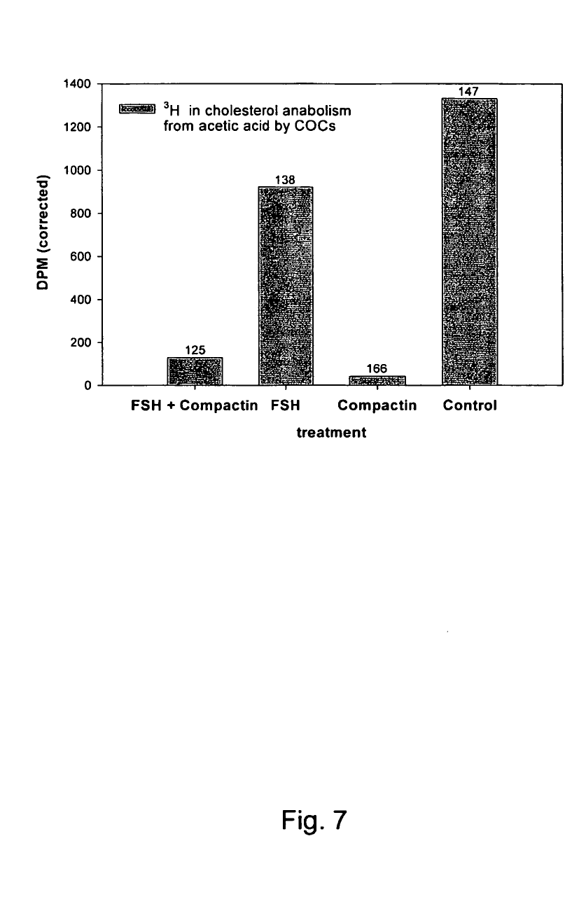

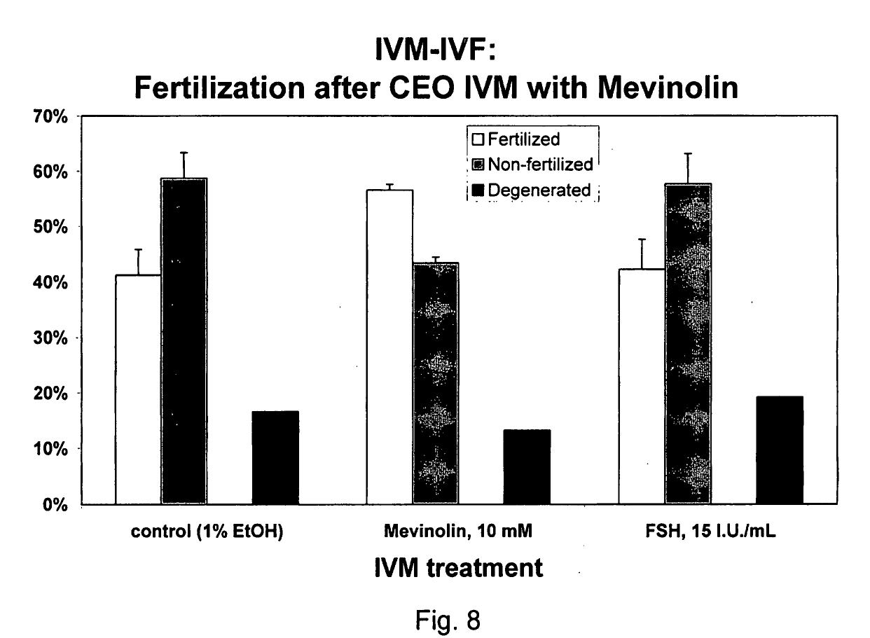

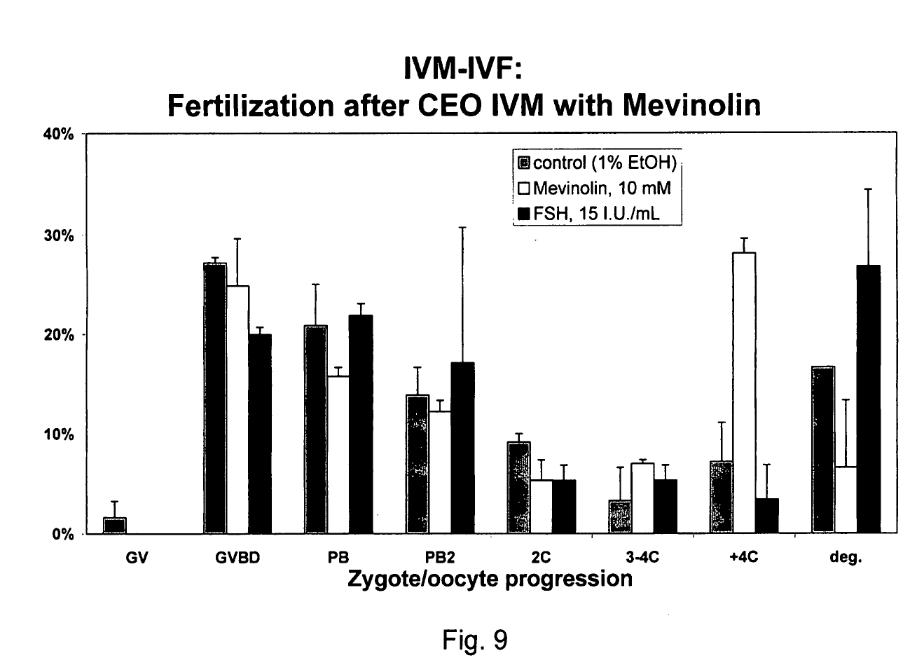

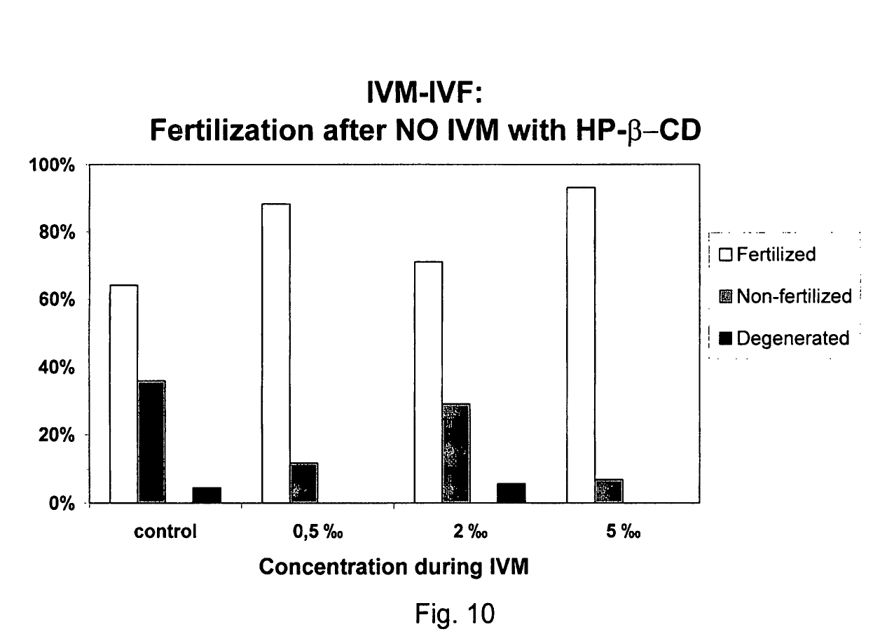

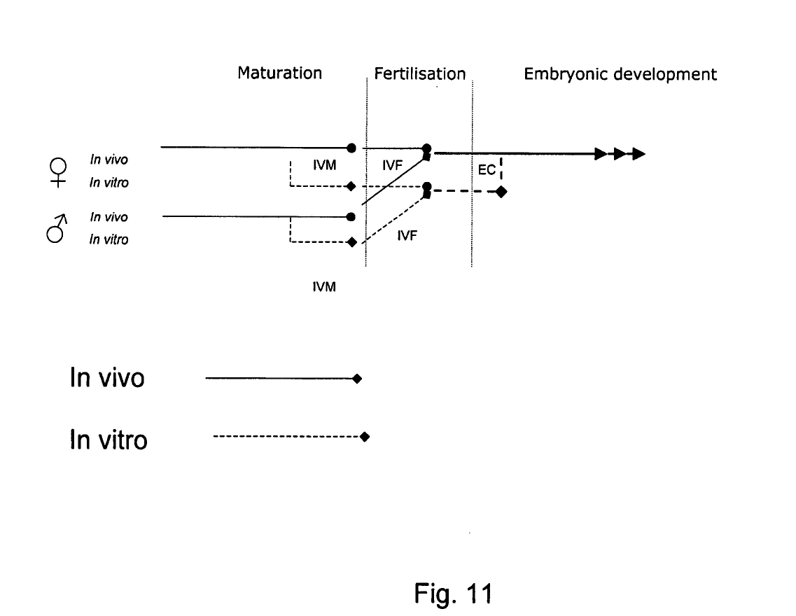

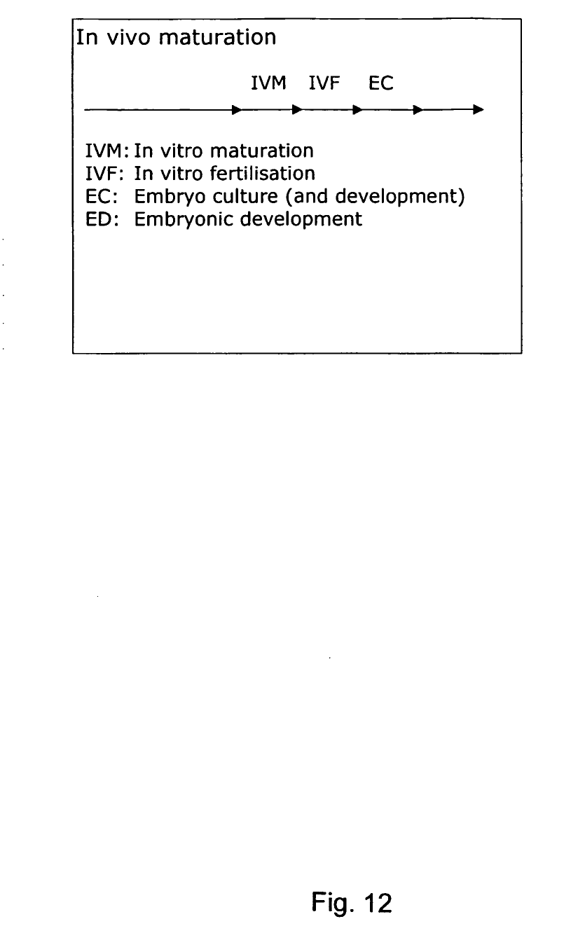

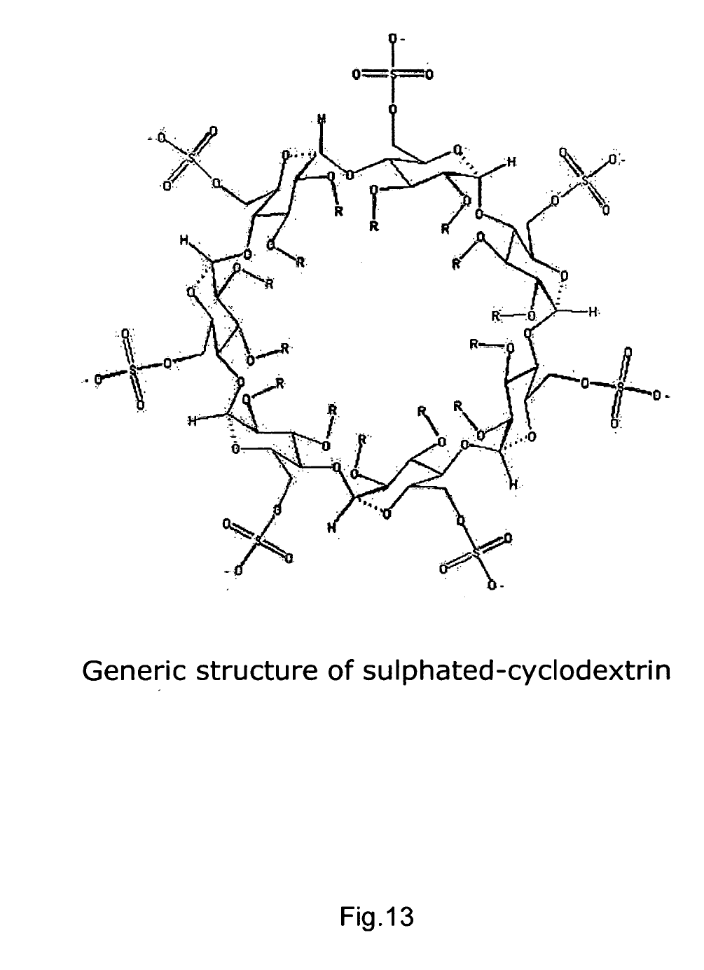

Dichotomy of the mammalian female and male germ cell development. One major dissimilarity between male and female germ cell development is indicated by the resting period. Male germ cells rest in the pre-meiotic stage whereas oocytes rest in the meiotic stage (prophase). The resting phase is the stage between embryonic development or early post-natal life and puberty, where the germ cells are undividing. Another major dissimilarity between male and female germ cells development in puberty is indicated by the male germ cell population renewal by the mitotic stem cell, in contrast to the female germ cell population being fixed in numbers.

FIG. 2

The mammalian de novo pre-sterol and sterol biosynthesis pathway. Pre-sterol biosynthesis ultimately starts with a condensation between acetate and coenzymeA to form acetyl-coenzymeA. Acetyl-coenzymeA is processed in a multienzymatic step, which may lead to the production of squalene. Squalene is converted by two enzymes into lanosterol, which is the root of the sterol biosynthesis pathway. The mammalian sterol biosynthesis in the endoplasmic reticulum involves at least the indicated 7 enzymes, but may in parallel to these involve a number of enzymes with similar catalytic characteristics. Enzymes are designated by italics on a grey background whereas sterol intermediates are indicated by ordinary letters with superscribed designations of the relevant C—C double bindings and non-cholesterol methyl substitutions. The bi-directional arrow indicates that the enzymatic step is reversible. The broken arrow between 24-enes (left-hand panel) and side-chain saturated sterols (right hand panel) indicates that the sterol delta24 reductase enzyme may have multiple sterol substrates.

FIG. 3

Branching of the pre-sterol biosynthesis pathway. The sterol metabolism indicated in FIG. 2 above is only one of several possible outcomes of the HMG-CoA-reductase mediated production of mevalonic acid. Two arrows between metabolites indicate a multi enzyme conversion.

FIG. 4

Structure, numbering of carbons and ring designation in the perhydrocyclopentanophenanthrene ring structure. C-numbering is in accordance with IUPAC convention.

FIG. 5

No of 2-cells in tuba of gonadotropin-stimulated prepubertal female mice after treatment with Mevacor®. The standard error of the mean is indicated by errorbars. The figure represents 5 independent experiments. The mean of all experiments is indicated on the right.

FIG. 6

Melotic maturation rate of mouse cumulus oocytes complexes (COC) after culture in FSH and statin (Compactin). GVBD represent oocytes in which no germinal vesicle was visible under a light microscope, which indicates that the oocyte has resumed meiosis. PB/GVBD indicates the ratio of polar body formation amongst the oocytes that resumed meiosis during the culture period. Each experiment was performed in quadruplicates and each experiment represents 30-40 COCs. Error bars indicate the standard error of the mean. Different letters above bars indicate a statistical difference at the 95% confidence level.

FIG. 7

Radio-tracing of ³H from acetic acid build in free cholesterol after 20 h of the above COC cultures shown in FIG. 6. DPM on the ordinate axis indicate the radioactivity measured by the scintillation detector after correction for counting efficiency and purification efficiencyby the HPLC method. N denotes the total number of COCs extracted for cholesterol isolation by HPLC.

FIGS. 8 and 9

Fertilisation rate of oocyte matured in vitro with a statin (Mevinolin) compared to control and FSH. The fraction of fertilised oocytes (PB²-oocytes to 4+ cells) increased by the addition of the statin Mevinolin to the IVM-medium of CEOs as compared to CEOs cultured in the presence of 7.5 I.U./L FSH and control CEOs. Moreover, (not shown) the Mevinolin treated oocytes displayed extensive cumulus expansion after the IVM culture period as opposed to the control oocytes. The FSH-treated oocytes were intermediate between these two groups in this respect. The degree of degeneration was not different between the groups. Also the development has reached a more progressed state in the Mevinolin treated group as compared to the two control groups after 72 hours of embryo culture.

FIG. 10

Fertilisation rate of oocyte matured in vitro with a cyclodextrin (hydroxypropyl-beta-cyclodextrin) compared to control. The fraction of fertilised oocytes (PB²oocytes to 4+ cells) increased by the addition of the sterol binding and transporting substance hydroxypropyl-cyclodextrin to the IVM-medium of NOs as compared to control NOs. The rate of degeneration was not affected by the presence of the cyclodextrin in the IVM-medium.

FIG. 11

Overview of the developmental processes that are be manipulated by the present invention. The present invention relates to the process of maturation and fertilisation both in vivo and in vitro and not to any embryonic development. The concept relating to inhibition of sterol biosynthesis de novo applies both in the male and in the female, whereas the concept relating to the use of sterol binding and transporting substances applies during maturation in the female only.

FIG. 12

Overview of the in vitro developmental processes described in the present application.

FIG. 13

The generic structure of sulphated-cyclodextrin. If R=CH2CH(OH)CH3 the structure represents hydroxypropyl-β-cyclodextrin used in example 8 below.

### EXAMPLES

### Example 1

Effect of a Statin Administration in Female Mice on the Number of 2-Cells in vivo After Fertilisation

Prepubertal C57 black×DBA 2 F1 female mice (M&B A/S, Ry, Denmark) were stimulated to super-ovulation (11 a.m. at day 0) by i.p. Injections of 12 IU Menogon® (Ferring, Denmark) in 200 μL H₂O. This gonadotrophin preparation contains 50% human FSH and 50% human LH in terms of biological activity. Half of the mice were given additional Injections of 1.6 mg lovastatin, by dissolving a tablet of Mevacor® (Merck Sharp and Dohme, NL) containing 40 mg lovastatin in 5 ml PBS and injecting 200 μL of the resulting slurry i.p. at three consecutive days, beginning at day 0. The other half (control mice) were injected a similar volume of PBS. At 1 p.m. at day 2 the animals were given an ovulatory dose of 10 IU hCG (Ferring, Denmark). The mice were caged individually one hour later with a mature male mouse of the same strain and supplier (1 male per female). Female and male mice were separated at 9 a.m. at day 3, females were left alone for another 48 h. After killing of the females and dissection of the tuba ovarii, 2-cells were flushed into a plastic dish with culture medium (see later) by mounting a glass pipette in one end of the isolated tuba and blowing by mouth through a filter. The total number of 2-cells flushed per mouse were hereafter counted. The 2-cells were subsequently cultured in Universal IVF-medium (Medicult, Denmark) in an incubator (37° C., 95% atmospheric air, 5% CO₂) in 4-well dishes (Nunclon 176740, Nunc, Denmark). After 4 days of culture, wells were scored and numbers of morula, blastocyst or degenerated entities were determined by use of a microscope:

The number of 2-cells per animal increased significantly when the mice were treated with 3×1.6 mg statin during the follicular phase maturation (FIG. 4). This represents the principal outcome with respect to reproduction of the cholesterol lowering treatment: the increase in the number of ovulated and fertilised oocytes as compared to control animals. The 2-cells were cultured in vitro and developed into blast-oocytes with recovery rates indiscriminate to controls.

### Example 2

Effect of a Statin Administration in Female Mice on the Number of 2-Cells in vivo After Fertilisation using Various Regimes

Prepubertal C57 black×DBA 2 F1 female mice (M&B A/S, Ry, Denmark) were treated as in example 1 but for a few deviations. In experiment A the dose of statins was decreased to 100 μg/animal and given as a single injection 15 minutes prior to the hCG-injection. In experiment B the dose of statins was decreased to 3×100 μg/animal and administered as in example 1. In experiment C the dose of statins was given as a single injection 15 minutes prior to the hCG-injectionin 9 month old mice. In experiment D the dose of gonadotropins were decreased gradually whereas the dose of statins was administered as in example 1.

Collectively, the experiments demonstrate that the dose of statins may be decreased to one tenth of the dose applied in example 1. Also, by use of appropriate concentrations the statin may be given as a single injection prior to the ovulatory stimulation by hCG as opposed to the application during a broader time window in the follicular phase. The add-on concept is demonstrated in experiment D where a lowered gonadotropin administration may be compensated for partly by the co-administration of a statin. All the experiments in the present example, however, need quantitative substantiation before conclusions are to made in these respects

TABLE 2
Number of 2-cells in C57 black × DBA 2 F1 mice after administration of
exogeneous gonadotropins and a statin (Mevacor).

| Exp. | | Gnrh | hCG | | Administration | No. of 2-cells in | Mated | Mating |
|---|---|---|---|---|---|---|---|---|
| no. | Animal stage | (I.U.) | (I.U.) | Statin treatment | regime | mated animals | no. | frequency |
| A | Pre-pubertal | 12 | 10 | — | | 22.0 ± 8.9 | 3 | 3 of 4 |
| | | | | 100 μg Mevacor | single, | 27.5 ± 3.5 | 2 | 2 of 4 |
| | | | | | ovulation | | | |
| | | | | 1000 μg Mevacor | single, | 15.3 ± 7.3 | 4 | 4 of 4 |
| | | | | | ovulation | | | |
| B | Pre-pubertal | 12 | 10 | — | | 9.5 ± 2.2 | 4 | 4 of 4 |
| | | | | 100 μg Mevacor | single, | 19.3 ± 4.1 | 4 | 4 of 6 |
| | | | | | ovulation | | | |
| | | | | 3 × 100 μg | multi, | 6.5 ± 1.0 | 4 | 4 of 5 |
| | | | | Mevacor | follicular | | | |
| | | | | | phase | | | |
| C | Mature, | 15 | 10 | — | | 25.2 ± 7.1 | 5 | 5 of 8 |
| | cycling | | | 1.0 mg Mevacor | single, | 16.8 ± 3.4 | 5 | 5 of 8 |
| | | | | | ovulation | | | |
| | | 12 | | — | | | | |
| | | 12 | | 3 × 1.0 mg | multi, | 11.4 ± 2.9 | 6 | 6 of 8 |
| | | | | Mevacor | follicular | | | |
| | | | | | phase | | | |
| D | Pre-pubertal | 4 | 5 | — | | 7.2 ± 1.4 | 6 | 6 of 8 |
| | | 4 | | 3 × 1.0 mg | multi, | 5 | 1 | 1 of 4 |
| | | | | Mevacor | follicular | | | |
| | | | | | phase | | | |
| | | 1.2 | | — | | 1 | 1 | 1 of 4 |
| | | 1.2 | | 3 × 1.0 mg | multi, | | 0 | 0 of 4 |
| | | | | Mevacor | follicular | | | |
| | | | | | phase | | | |
| | | | | | | | 0 | 0 of 4 |

### Example 3

Administration of a Statin Augments the LH/hCG Induced Decrease in the Tissue Density of Free Cholesterol in Ovaries After Gonadal Stimulation of Pre-Pubertal Mice

Prepubertal C57 black×DBA 2 F1 female mice (M&B A/S, Denmark) were stimulated to super-ovulation (11 a.m. at day 0) by i.p. injections of 12 IU Menogon® (Ferring, Denmark) in 200 μL H₂O (as in example 1). Half of the mice were given additional injections of 1.6 mg lovastatin, by dissolving a tablet of Mevacor® (Merck Sharp and Dohme, NL) containing 40 mg lovastatin in 5 ml PBS and injecting 200 μL of the resulting slurry i.p. at three consecutive days, beginning at day 0 (as in example 1). The other half (control mice) were injected a similar volume of PBS. At 1 p.m. at day 2 the animals were given an ovulatory dose of 10 IU hCG (Ferring, Denmark) and killed 6 h after for preparation and HPLC-analysis of ovarian extracts: Ovaries were isolated pair-wise, weighed, freeze-dried, weighed again and subsequently extracted in 1.0 ml (v/v) 75% n-heptane: 25% isopropanol. The organic extract was re-constituted in mobilphase for HPLC straight-phase (SP) separation (ChromSpherSi, 5 μm, 250×4.6 mm HPLC column, running in (v/v) 99.5% n-heptane (Fischer, Leicestershire, U.K.): 0.5% isopropanol (Baker, NL) at 1.00 ml/min.). Eluted material was detected by ultraviolet light absorption between 200-300 nm. Prior to the HPLC analysis a standard mixture containing cholesterol, FF-MAS and progesterone (P₄) was run three times in order to confirm the stability of response factors for standards. Standards for cholesterol (Steraloids C6760) and P₄ (Sigma P-0130) were obtained commercially whereas the FF-MAS standards used was produced in the laboratory as described before (Baltsen et al., 1999). All standard curves were linear in the range of all sample values.

TABLE 3
Free cholesterol (C), follicular fluid meiosis-activating sterol
(FF-MAS) and progesterone (P₄) in gonadotrophin primed prepubertal
mouse ovaries stimulated with hCG and statin. Different post-
designation letters (boldfaced) denote statistical difference by a t-test.

| Stimulation group | hCG + statin | hCG | — |
|---|---|---|---|
| N | 13 | 6 | 6 |
| mouse weigh (g) | 14.6 ± 0.2 A | 15.3 ± 0.3 A | 14.6 ± 0.3 A |
| N | 5 | 5 | 3 |
| ovarian weigh (mg) | 2.3 ± 0.1 A | 2.2 ± 0.2 AB | 1.9 ± 0.1 B |
| C, ovary (mg/g | 2.2 ± 0.1 A | 2.7 ± 0.1 B | 3.0 ± 0.2 B |
| wet w.) | | | |
| C, ovary (mg/g dry w.) | 10.5 ± 0.6 A^{§} | 11.0 ± 0.4 A^{§} | 12.1 ± 0.5 A |
| C, liver (mg/g wet w.) | 1.7 ± 0.1 A^{#} | (not assayed) | 2.9 ± 0.3 B |
| C, liver (mg/g dry w.) | 6.5 ± 0.5 A^{#} | (not assayed) | 7.1 ± 0.4 A |
| FF-MAS (μg/g wet w.) | 0.13 ± 0.05 A | 3.0 ± 0.5 B | 0.76 ± 0.66 A |
| P₄ (μg/g wet w.) | 14.9 ± 0.9 A | 13.0 ± 0.5 A | 1.3 ± 0.7 B |

^{#}N = 3.
^{§}Borderline significance (P = 0.055 with the alternative hypothesis that “hCG + statin” < “control”). Borderline significance (P = 0.061 with the alternative hypothesis that “hCG” < “control”).

The tissue density of free cholesterol decreases in the mouse ovary prior to ovulation. The tendency of decreasing tissue density in the ovaries of free cholesterol after hCG stimulation was pronounced by the statin treatment. The decrease can not be completely explained by the oedematic effect of the steroid generating hCG hormone in that the cholesterol tissue density also decreased when evaluated on the basis of dry tissue weights. HCG was applied in order to mimic the natural pre-ovulatory LH-surge. It is important to note that the extent of progesterone (steroid) production was not influenced by the statin treatment. Also, it should be noted that the statin completely blocks the LH/hCG induced accumulation of the pre-cholesterol metabolite FF-MAS and leaves the ovary with a level of FF-MAS below the mice that was not challenged with hCG. Moreover, comparing control and hCG-stimulated mouse ovaries reveal that the absolute decrease of cholesterol is >100 times the increase in FF-MAS with respect to the tissue density.

### Example 4

A HMG-CoA-Reductase Inhibitor Stimulates Maturation of Cumulus Enclosed Oocytes in vitro and Augments Gonadotrophin Stimulated Maturation of Cumulus Enclosed Oocytes in vitro

Prepubertal C57 black×DBA 2 F1 female mice (M&B A/S, Ry, Denmark) were stimulated to super-ovulation (11 a.m. at day 0) by i.p. injections of 12 IU Menogon® (Ferring, Denmark) in 200 μL H₂O as in examples 1 and 2. Oocytes from preovulatory follicles were retrieved from the ovaries 46 hours after by puncturing the pre-ovulatory follicles of the ovaries with 0.20 mm needles when ovaries were immersed in a culture medium (α-MEM 22571-020, Gibco BRL, Scotland) supplemented with 4 mM hypoxanthine (see below). Cumulus oocyte complexes (COC) were collected by identification through a preparation microscope and subsequent sucking through a membrane mounted mouth pipette and cultured in α-MEM in an incubator (37° C., 95% atmospheric air: 5% CO₂) in 4-well dishes (Nunc, Denmark). The culture medium was supplemented with 4 mM hypoxanthine (HX) (Sigma H-9377, Sigma-Aldrich, Denmark), 200 mM L-glutamine (Gibco BRL, Scotland), 20000 IU/L penicillin (Gibco BRL, Scotland), 20000 IU/L streptomycin (Gibco BRL, Scotland) and 3 mg/ml BSA (Sigma A-7030, Sigma-Aldrich, Denmark). All incubations were also added 25 mCl ³H-acetat (NEN, U.S.A.) in order to measure de novo synthesis of cholesterol after incubation by scintillation. The statin Compactin (Sigma, Sigma-Aldrich, Denmark,) was added in 50 mM in DMSO (final concentration of DMSO in cultures were 1% v/v in all set-ups including controls) and recombinant human follicular stimulating hormone (Gonal-F, Serono, DK) was added to a final concentration of 7.5 IU/L. After 22 hours of culture oocytes were scored for the presence of germinal vesicles (GV), germinal vesicle breakdown (GVB) and presence of polar bodies (PB). The three states represent a progression of maturation of the oocyte.

There was a melotic maturing effect of the cholesterol-lowering regime per se that appeared to be additive to the well known maturing effect of FSH (FIG. 4). Pooling the cells from the quadruplicates and measuring the amount of ³H metabolised from acetic acid verified that the de novo cholesterol biosynthesis was indeed decreased by addition of the statin (FIG. 5). The effect of Compactin was pronounced whereas the effect of FSH was less effective in inhibition of cholesterol de novo biosynthesis. This experiment confirms that the inhibition of cholesterol de novo synthesis by use of statin potentates the natural maturational events caused by gonadotrophins in vitro and that the blocking of the de novo synthesis of sterols is enhanced at the same time.

### Example 5

hCG Mediates a Ceasing De Novo Bosynthesis of Cholesterol in Mouse Ovaries in vitro

Prepubertal C57 black×DBA 2 F1 female mice (M&B A/S, Ry, Denmark) were stimulated to super-ovulation (day 0) by i.p. injections of 12 IU Menogon® (Ferring, Vanløse, Denmark) in 200 μL H₂O as in examples 1-4. Ovaries were isolated 46 hours after as in example 4 and cultured in toto in α-MEM (Gibco BRL, Scotland) in presence of 0.25 mCi ³H-acetic acid (NEN, U.S.A.). Cultures were added 0 (control) or 5 IU/ml hCG and 0 (control) or 20% FCS in combinations. 2 ovaries were incubated together in 1.0 ml medium. 22 hours after, cultures were stopped, ovaries rinsed and extracted as explained in example 3. The extracted sterols were separated by HPLC Column: ChromSpherSi 250×4.6 mm, 5 μm; mobile phase: 99.65% n-heptane (Fisher, Leicester, U.K.): 0.35% isopropanol (Baker, Deventer, NL) (vol.:vol.)), regeneration; 90% n-heptane: 10% isopropanol. Prior to straight phase sample analysis three consecutive runs of a standard mixture containing squalene, 4,4-dimethylsterols, 7-dehydrocholesterol, desmosterol, cholesterol and P₄ were performed in order to tabulate time windows for collection. Window 1 (2′50″-4′00″) containing squalene, window 2 (10′30″-14′00″) containing 4,4-dimethylsterols, window 3 (25′00″-29′00″) containing cholesterol and desmosterol, window 4 (29′30″-33′00″) containing 5,7-enes and window 5 (containing P₄) were collected, dried and subjected to reversed phase: LiChrospher RP-8, 5 μm, 250×4.6 mm HPLC column running in (v/v) 92.5% acetonitrile: 7.5% water at 1.00 ml/min, 40° C. Analytes were identified by UV light absorption between 200-300 nm. Squalene (Merck S21362), lanosterol (Sigma L5768), zymosterol (Steraloids C3200), lathosterol (Steraloids C7400), 7-dehydrocholesterol (Steraloids C3000), desmosterol (Steraloids C3150), cholesterol (Steraloids C6760) and P₄ (Sigma P-0130) were obtained commercially, whereas FF-MAS and T-MAS standards were produced in the laboratory. All the below ranked analytes eluted as single homogeneous single peaks on the reversed phase separation. Peaks were collected, mixed with 3.0 ml scintillation fluid (Packard, NL) in scintillation vials. Countings were performed in a Beckman LS 1801 liquid scintillation counter. CPM values were corrected for counting efficiency and sample injection:

TABLE 4
Scintillation data for analytes separated by HPLC after incubation
in vitro of mouse ovaries in presence of ³H-acetic acid

| Analyte | Control/hCG | P | Control/FCS | P |
|---|---|---|---|---|
| Squalene | 80 ± 9% | 0.099 | 136 ± 20% | 0.139 n.s. |
| Lanosterol | 22 ± 7% | 0.000074 | 331 ± 98% | 0.065 n.s. |
| FF-MAS | 72 ± 16% | 0.133 | 63 ± 11% | 0.021** |
| T-MAS | 43 ± 50% | 0.000059 | 96 ± 13% | 0.76 n.s. |
| 5,7-ene | 35 ± 4% | 0.000071 | 161 ± 33% | 0.142 n.s. |
| Desmosterol | 58 ± 7% | 0.0041 | 91 ± 13% | 0.55 n.s. |
| Cholesterol | 226 ± 39% | 0.032* | 111 ± 32% | 0.75 n.s. |
| Progesterone | 13 ± 5% | 0.000085 | — | n.c.*** |

P-values result from a paired test of 6 + 6 samples with the hypothesis that control values are non-equal to test values.
*Decrease after addition of hCG.
**Increase with addition of FCS.
***Not compared due to low scintillation figures.
n.s. denotes non-significance.

This experiment confirms that the de novo biosynthesis of cholesterol decreases in mouse ovaries following hCG treatment whereas the accumulation of pre-cholesterol sterols produced de novo increase. Also, the amount of radioactivity associated to progesterone, the major steroid produced in ovaries after hCG/LH treatment, decreased. This reveals that the total amount of substrate arising from de novo produced cholesterol decreases when ovaries are stimulated to ovulate. The fact that the amount of radioactivity incorporation into the various sterol fraction was independent of lipoprotein (FCS) supplementation indicate that the biological mechanism of cholesterol lowering in the ovaries following the ovulatory stimulation was not influenced by the usual cholesterol homeostatic mechanisms.

### Example 6

Changing the Ratio of Cholesterol to Phospholipids (C/PL-ratio) in the Motile Cell Fraction of Epididymis in Mice by Prior Administration of a Statin

Post-pubertal C57 black×DBA 2 F1 male mice (M&B A/S, Ry, Denmark) are used. Four mice are given 3 i.p. injections of 1.0 mg lovastatin per 30 g body weight by dissolving a tablet of Mevacor® (Merck Sharp and Dohme, NL) containing 40 mg lovastatin in 8 ml PBS and injecting 200 μL of the resulting slurry i.p. at 1 p.m. at day-6, -3 and -1, counting the day of epididymal sperm harvest as day 0. Four other mice (control mice) are injected a similar volume of PBS. At 10 a.m on day 0 all mice are killed and the epididymis are prepared in physiological saline. Thereafter, the epididymis are transferred to a 1-mL culture well containing MEM (Gibco BRL, Scotland) supplemented with 2.3 mM pyruvate, 200 mM L-glutamine (Gibco BRL, Scotland), 20000 IU/L penicillin (Gibco BRL, Scotland), 20000 IU/L streptomycin (Gibco BRL, Scotland) and 0.2 mg/ml BSA (Sigma A-7030, Sigma-Aldrich, Denmark) and placed in an incubator (37° C., 95% atmospheric air: 5% CO₂).

After 1.5 hours of incubation, the upper liquid phase is aspirated and transferred to a new glass-vial and centrifuged. After centrifugation, the supernatant is discarded and the pellet is freeze-dried and thereafter extracted in 50% methanol:50% chloroform (vol./vol.). The extract is subsequently divided in two equal parts. One half is subjected to sterol analysis as designated in the examples above and the total sterol content is calculated to consist of cholesterol, desmosterol, cholesta-7,24-dien-3β-ol, T-MAS and lanosterol. The other half is subjected to phospholipid assay according to the method of Bartlett (Bartlett, 1958). The C/PL-ratio is calculated for each individual animal.

It is contemplated that the C/PL-ratios in the statin treated group is lower the C/PL-ratios in the control group.

### Example 7

Statins Applied During IVM Increase the Fertilisation Rate in Mouse Cumulus Enclosed Oocytes (CEO) Following IVF

Mouse oocytes were isolated and cultured according to descriptions in example 3 but for a single deviation in that the IVM culture medium was further supplemented with 1 mg/mL Fetuin (Sigma F-3385, Sigma, U.S.A.). The CEOs were divided into three groups and cultured in dishes containing medium added either 1% (v/v) ethanol (control), recombinant human follicular stimulating hormone (Gonal-F, Serono, DK) at a concentration of 7.5 IU/L together with 1% (v/v) ethanol (FSH), or 10 μM Compactin (Mevinolin M2147, Sigma-Aldrich, Denmark) with 1% (v/v) ethanol (Mevinolin). The ethanol was necessary in order to dissolve Mevinolin in the medium. After in vitro maturation for 22 h, oocytes were transferred to a fertilisationn medium consisting of minimal essential medium (MEM) (21090-022, Gibco BRL, Scotland), 200 mM L-glutamine (Gibco BRL, Scotland), 20000 IU/L penicillin (Gibco BRL, Scotland), 20000 IU/L streptomycin (Gibco BRL, Scotland), 3 mg/ml BSA (Sigma A-7030, Sigma-Aldrich, Denmark), 2.3 mM sodium pyruvate (S-8636, Sigma), 10 mM ethylen diamine tetra acetic acid (EDTA, Merck 1.08418.0250) and 1 mg/mL Fetuin (Sigma F-3385, Sigma, U.S.A.). Prior to oocyte transfer into the fertilisation medium, mouse sperm for IVF was obtained the following way: A 6-9 month old C57 black×DBA 2 F1 male mouse (M&B A/S, Ry, Denmark) was killed and the epididymis prepared in physiological saline. Thereafter, the epididymis were cut in small pieces and the bulk material was transferred to four 1-mL culture well containing the 800 μL IVF-medium described above and subsequently placed in an incubator (37° C., 95% atmospheric air: 5% CO₂). After 1.5 hours of incubation, 300 μL of the upper liquid phase of each of the four wells was aspirated and transferred to a new glass-vial. The resulting pool of motile sperm was counted in a counting chamber and 50000 motile sperm cells was added each oocyte culture. The IVF cultures were incubated at incubator 37° C., 95% atmospheric air: 5% CO₂ for 20 hours after which the oocytes were transferred to a fresh IVF medium equilibrated under the same conditions. These oocytes were left for another 48 hours and subsequently counted according to the maturational status of the oocyte or the progression of the fertilised zygote, i.e. GV-oocytes, GVBD-oocytes, PB-oocytes, PB²-oocytes (oocytes with a visible second polar body), 2-cells, 3-4-cells, 4+ cells (zygotes with more than 4 blastomeres) and degenerated cells (usually granulated or cells with extensive plasma-membrane ondulation).

### Example 8

Cyclodextrin Applied During IVM Increase the Fertilisation Rate in Naked Mouse Oocytes (NO) Following IVF

Mouse oocytes were isolated and cultured according to descriptions in example??. The NOs were divided into three groups and cultured in dishes containing medium added either 0%o, 0.5%o, 2.0%o or 5%o hydroxypropyl-cyclodextrin (ICN 153540, ICN Ohio, U.S.A.). IVM and IVF conditions were the same as in example 6.

### Example 9

Increasing Sperm Quality in Mice by Prior Administration of a Statin

The set-up involves the same animals and statin administration as in example 6. The animals are analysed by methods that are developed in order to evaluate the fertilising potential and sperm quality. It is contemplated that statin treated animals will have a fertility premium as compared to the control animals.

References

Awano et al. 1989, Lipids, 24(7):662-664.
Baltsen et al., 1999 Biomed Chromatogr, 13(6):382-388.
Baltsen et al., 1998, Journal of Reproduction and Fertility Abstract Series, 21:16.
Bartlett, 1958, J Biol Chem, 234:466-468.
Benoff et al., 1993a, Hum Reprod, 8(12):2141-2154.
Benoff et al., 1993b, Hum Reprod, 8(12):2155-2166.
Bernini et al., 1998, J Endocrinol Invest, 21(5):310-317.
Connor et al., 1997, Invest Opthal Vis Sci, 38(12):2619-2628.
Cross, 1996, Biol Reprod, 54(1): 138-145.
Cross, 1998, Biol Reprod, 59(1):7-11.
Davis, Arch Androl, 1980, 5(3):249-254.
Davis, 1981, Proc Natl Acad Sci U.S.A., 78(12):7560-7564.
Gamzu et al., 1997, Biol Reprod, 539-546.
Go et al., 1985, Bio Reprod, 32:145-153.
Goldstein et al., 1990, Nature 343: 425-430.
Gunasageram et al., 1995, Med Sci Res, 23:315-316.
Hamamah et al., 1995, Andrlogia, 27(2):91-97.
Huacuja et al., 1981, Arch Androl, 7(4):343-349.
Lopez et al., 1991, Mol Reprod Dev 28(2):158-168.
Moya et al., 1994, Arterioscler. Thromb., 14(7):1056-1065.
Sugkraroek et al., 1991, Fertil Steril, 55:820-827.
Yamamoto et al., 1999, Hum Reprod, 14(6):1516-1521.

The invention will now be further described by the following numbered paragraphs:
1. A method for inhibiting the sterol de novo biosynthesis in the cells of the mammalian gonad and/or at least one mammalian germ cell and/or gamete and/or gonadal derived cell supporting germ cells with sterols, thereby increasing the developmental competence of at least one mammalian gamete, zygote, early embryo, implanted blastocyst and/or embryo, and/or increasing the number of mammalian gametes with fertilisation capability, resulting in an increased number of zygotes, early embryos, implanted blastocysts and/or embryos per reproductive cycle in the female after mating, the method comprising administering a compound or a combination of compounds capable of inhibiting the sterol de novo biosynthesis of a mammal in need thereof.
2. A method for increasing the sterol efflux from at least one mammalian ovary and/or mammalian oocyte and/or mammalian female gamete and/or ovary derived cell surrounding an oocyte, thereby increasing the developmental competence of said at least one mammalian ovary and/or mammalian oocyte and/or mammalian female gamete and/or ovary derived cell surrounding an oocyte, the method comprising administering a compound or a combination of compounds capable of promoting the sterol efflux to a mammal in need thereof.
3. A method according to paragraph 1 or 2, wherein the mammal in the need thereof is female and the inhibition and/or efflux takes place during a period of 0-12 days before ovulation and/or 0-3 days after ovulation.
4. A method according to paragraph 1, wherein the mammal in the need thereof is male and the inhibition takes place during a period of 0-12 days before ejaculation.
5. A method for increasing the developmental competence of at least one mammalian gamete, zygote, early embryo, implanted blastocyst and/or embryo and/or for increasing the number of mammalian gametes with fertilisation capability, resulting in an increased number of zygotes, early embryos, implanted blastocysts and/or embryos per reproductive cycle in the female, the method comprising adding a compound or a combination of compounds capable of inhibiting the sterol de novo biosynthesis of said at least one mammalian gamete, zygote, early embryo, implanted blastocyst and/or embryo to an in vitro medium which supports the respiration and/or maturation of at least one oocyte and/or at least one spermatozoon.
6. A method for increasing the sterol efflux from at least one mammalian ovary, oocyte, female gamete, and/or ovary derived cell surrounding an oocyte thereby increasing the developmental competence of said at least one mammalian ovary and/or mammalian oocyte and/or mammalian female gamete and/or ovary derived cell surrounding an oocyte, the method comprising adding a compound or a combination of compounds capable of promoting the sterol efflux to an in vitro medium which supports the respiration and/or maturation of at least one oocyte.
7. A method according to any of paragraphs 1, or 3-5, wherein the compound is a substance or a combination of substances that antagonises one or more of the mammalian enzymes involved in the biosynthesis pathway between acetyl-coenzyme A and lanosterol, the enzymes being HMG-COA reductase, mevalonate kinase, phosphomevalonate kinase, pyrophosphomevalonate decarboxylase, isopentenyl pyrophosphate isomerase, dimethylallyl transferase, geranyl transferase, squalene synthase, squalene monoxygenase, oxidosqualene lanosterol cyclase and lanosterol synthase.
8. A method according to any of paragraphs 1, 3-5 or 7, wherein the compound is a substance or a combination of substances that antagonises the 3-hydroxy-3-methylglutaryl coenzyme A reductase enzyme (HMG-COA reductase).
9. A method according to any of paragraphsl, 3-5 or 7-8, wherein the compound is a statin or a combination of statins.
10. A method according to any of paragraphs 1, 3-5 or 7-9, wherein the statin is selected from the group consisting of atorvastatin, cerivastatin, fluvastatin, BAY W 62, pravastatin, lovastatin (formerly called mevinolin or monacolin K), 28, HR 780, pravastatin, simvastatin, compatin, methyl-compactin, mevastatin (formerly called campactin or ML-236B), ML-236A, ML-236C, dihydrocompactin, monacolin J, monacolin L, monacolin M, monacolin X, dihydromonacolin L, mevinolin, 3β-hydroxycompactin acid monosodium salt (pravastatin, CS-514), synvinolin, cerivastatin and nystatin/triamcin.
11. A method according to any of paragraphs 2-3 or 6, wherein the compound or a combination of compounds capable of promoting the sterol efflux is selected from the group consisting of cyclodextrin, chemically modified cyclodextrins, high densityliproprotein (HDL), an apoprotein derived from HDL, and sterol carrier protein I and II.
12. A method according to any of paragraphs 2-3, 6 or 11, wherein the compound is cyclodextrin.
13. A method according to any of the preceding paragraphs, wherein the amount of statin and/or compound capable of promoting the sterol efflux administered is between 0.01-100 mg per kg body weight per day.
14. A method according to any of the preceding paragraphs, wherein the amount of statin and/or compound capable of promoting the sterol efflux administered to an in vitro medium is 0.01-1000 μM.
15. A method according to any of the preceding paragraphs, wherein the culturing of at least one oocyte in an in vitro medium is between 1 second and 15 days.
16. A method according to any of paragraphs 1, 3-5, 7-10 or 13-15, wherein the culturing of a spermatozoa in an in vitro medium is between 1 second and 8 days.
17. A method according to any of the preceding paragraphs, wherein the administration and/or addition is combined with an administration and/or addition of a gonadotrophin
18. A method according to any of the preceding paragraphs, wherein the administration and/or addition is combined with an administration and/or addition of a mammalian growth factor.
19. A method according to paragraph 17, wherein the amount of gonadotrophin administered in vitro is between 0-1000 IU/L.
20. A method according to paragraph 17, wherein the amount of gonadotrophin administered in vivo is between 0-5 IU FSH per kg body weight per day and 0-200 IU LH per kg body weight per day.
21. A method according to any of the preceding paragraphs, wherein the mammal is selected from the group consisting of human, horse, cow, rat, mouse, pig, sheep, goat, llama, dog, cat and mink.
22. A method according to any of the preceding paragraphs, wherein the mammalian gonadal cell is a human cell that support a germ cell with sterols or an isolated germ cell.
23. A method according to any of the preceding paragraphs, wherein the mammalian gonadal cell is an cumulus enclosed oocyte or an isolated oocyte.
24. A method according to any of paragraphs 1, 3-5, 7-10 or 13-23, wherein the germ cell is an ejaculated spermatozoa.
25. A method according to any of the preceding paragraphs, wherein the mammalian gonadal cell is an immature oocyte with its adhering cumulus cells and/or an isolated immature oocyte retrieved from large antral follicles or an oocyte with its adhering cumulus cells and/or an immature oocyte retrieved from pre-antral follicles, small or medium sized antral follicles, a small human antral follicle being 0.4-<5 mm in diameter, a medium sized human antral follicle being 5-<15 mm and a large human antral follicle being 15 mm or above in diameter.
26. A method according to any of the preceding paragraphs, wherein the gonadal cell is an immature or mature oocyte obtained after culture of at least one immature follicle from a stage between the primordial and the preantral stage.
27. A cell culture medium comprising a statin or a combination of statins.
28. A cell culture medium comprising a compound or combination of compounds capable of promoting the sterol efflux, such as a cyclodextrin.
29. A cell culture medium comprising a statin and a cyclodextrin.
30. A cell culture medium according to any of paragraphs 27-29, wherein said medium is a chemically defined cell culture medium.
31. A cell culture medium according to any of paragraphs 27-30, further comprising at least one growth factor.
1. 32. A cell culture medium according to any of paragraphs 27-31, wherein said growth factor is selected from the group consisting of gonadotropin (FSH, LH, CG), IGF, EGF, Insulin, growth hormone, interleukines and other peptid factors.
33. A method for maturating an immature oocyte obtained from a stage between the primordial follicle and the pre-antral stage comprising culturing said immature oocyte in a cell culture medium according to any of paragraphs 27-32.
34. A pharmaceutical composition comprising a combination of a compound capable of inhibiting the sterol de novo biosynthesis and compound capable of increasing the sterol efflux in a combination together with a pharmaceutically acceptable carrier.
35. A pharmaceutical composition according to paragraph 34, wherein said compounds are a statin and a cyclodextrin.
36. A pharmaceutical composition according to any of paragraphs 34-35, further comprising a growth factor.
37. A pharmaceutical composition according to paragraph 36, wherein said growth factor is selected from the group consisting gonadotropin (FSH, LH, CG), IGF, EGF, Insulin, growth hormone, interleukines and other peptid factors.
38. A pharmaceutical composition comprising a combination of a compound capable of inhibiting the sterol de novo biosynthesis and a growth factor.
39. A pharmaceutical composition according to paragraph 38, wherein said compound is a statin.
40. A pharmaceutical composition comprising a combination of a compound capable of promoting the sterol efflux and a growth factor.
41. A pharmaceutical composition according to paragraph 40, wherein said compound is a cyclodextrin.
42. A pharmaceutical composition according to paragraph 38 or 40, wherein said growth factor is selected from the group consisting of gonadotropin (FSH, LH, CG), IGF, EGF, Insulin, growth hormone, interleukines and other peptid factors
43. Use of a compound or a combination of compounds, capable of inhibiting the sterol de novo biosynthesis in mammalian gonads and/or at least one mammalian germ cell and/or gamete and/or gonadal derived cell, for the preparation of a medicament for inhibiting the sterol de novo biosynthesis in at least one mammalian germ cell and/or gamete and/or gonadal derived cell supporting germ cells with sterols, thereby increasing the developmental competence of at least one mammalian gamete, zygote, early embryo, implanted blastocyst and/or embryo, and/or for increasing the number of mammalian gametes with fertilisation capacity, resulting in an increased number of zygotes, early embryos, implanted blastocysts and/or embryos per reproductive cycle in a female after mating.
44. Use of a compound or a combination of compounds, capable of promoting the sterol efflux from at least one mammalian ovary and/or mammalian oocyte and/or mammalian female gamete and/or ovary derived cell surrounding an oocyte, for the preparation of a medicament for promoting the sterol efflux in at least one mammalian ovary and/or mammalian oocyte and/or mammalian female gamete and/or ovary derived cell surrounding an oocyte, thereby increasing the developmental competence of at least one mammalian ovary and/or mammalian oocyte and/or mammalian female gamete and/or ovary derived cell surrounding an oocyte.
45. Use according to paragraph 43 or 44, wherein the mammal is a female and the inhibition takes place during a period of 0-12 days before ovulation and/or 0-3 days after ovulation.
46. Use according to paragraph 43, wherein the mammal is a male and the inhibition takes place during a period of 0-12 days before the ejaculation.
47. Use of a compound or a combination of compounds, capable of inhibiting the sterol de novo biosynthesis, for the preparation of an in vitro medium which supports the respiration and/or maturation of at least one oocyte and/or at least one spermatozoo, for increasing the developmental competence of at least one mammalian gamete, zygote, early embryo, implanted blastocyst and/or embryo, and/or for increasing the number of mammalian gametes with fertilisation capacity, resulting in an increased number of zygotes, early embryos, implanted blastocysts and/or embryos per reproductive cycle in a female.
48. Use of a compound or a combination of compounds, capable of promoting the sterol efflux, for the preparation of an in vitro medium which supports the respiration and/or maturation at least one oocyte, for increasing the developmental competence of at least one mammalian ovary and/or mammalian oocyte and/or mammalian female gamete and/or ovary derived cell surrounding an oocyte
49. Use according to any of paragraphs 43 or 45-47, wherein the compound is a substance or a combination of substances that antagonises one or more of the mammalian enzymes involved in the biosynthesis pathway between acetyl-coenzyme-A and lanosterol, the enzymes being the 3-hydroxy-3-methylglutaryl coenzyme A reductase enzyme (HMG-CoA reductase), mevalonate kinase, phosphomelovanate kinase, pyrophosphomevalonate decarboxylase, isopehtenyl pyrophosphate isomerase, dimethylallyl transferase, geranyl transferase, squalene synthase, squalene monoxygenase, oxidosqualene lanosterol cyclase and lanosterol synthase.
50. Use according to any of paragraphs 43, 45-47 or 49, wherein the compound is a substance or a combination of substances that antagonises the 3-hydroxy-3-methylglutaryl coenzyme A reductase enzyme (HMG-COA reductase).
51. Use according to any of paragraphs 43, 45-47 or 49-50, wherein the compound is a statin or a combination of statins.
52. Use according to any of paragraphs 43, 45-47 or 49-51, wherein the statin is selected from the group consisting of atorvastatin, cerivastatin, fluvastatin, BAY W 62, pravastatin, lovastatin (formerly called mevinolin or monacolin K), 28, HR 780, pravastatin, simvastatin, compatin, methyl-compactin, mevastatin (formerly called campactin or ML-236B), ML-236A, ML-236C, dihydrocompactin, monacolin J, monacolin L, monacolin M, monacolin X, dihydromonacolin L, mevinolin, 3β-hydroxycompactin acid monosodium salt (pravastatin, CS-514), synvinolin, cerivastatin and nystatin/triamcin.
53. Use according to any of paragraphs 44-45 or 48, wherein the compound or combination of compounds capable of promoting the sterol efflux is selected from the group consisting of cyclodextrin, chemically modified cyclodextrins, high densityliproprotein (HDL), an apoprotein derived from HDL, and sterol carrier protein I and II.
54. Use according to any of paragraphs 44-45, 48 or 53, wherein the compound is cyclodextrin.
55. Use according to any of the preceding paragraphs, wherein the amount of statin to be administered is between 0.01-100 mg per kg body weigh per day.
56. Use according to any of the preceding paragraphs, wherein the amount of statin and/or compound capable of promoting the sterol efflux to be administered to an in vitro medium is 0.01-1000 μM.
57. Use according to any of the preceding paragraphs, wherein the culturing of at least one oocyte in an in vitro medium is between 1 second and 15 days.
58. Use according to any of paragraphs 43, 45-47, 49-52 or 55-57, wherein the culturing of a spermatozoa in an in vitro medium is between 1 second and 8 days
59. Use according to any of the preceding paragraphs, wherein the administration and/or addition is combined with an administration and/or addition of a gonadotrophin.
60. Use according to any of the preceding paragraphs, wherein the administration and/or addition is combined with an administration and/or addition of a mammalian growth factor.
61. Use according to paragraph 59, wherein the amount of gonadotrophin administrated in vitro is between 0-1000 IU/L.
62. Use according to paragraph 59, wherein the amount of gonadotrophin administrated in vivo is between 0-5 IU FSH per kg body weight per day and 0-200 IU LH per kg body weight per day.
63. Use according to any of the preceding paragraphs, wherein the mammal is selected from the group consisting of human, horse, cow, rat, mouse, pig, sheep, goat, llama, dog, cat and mink.
64. Use according to any of the preceding paragraphs, wherein the mammalian gonadal cell is a human cell that supports a germ cell with sterols or an isolated germ cell.
65. Use according to any of the preceding paragraphs, wherein the mammalian gonadal cell is a cumulus enclosed oocyte or an isolated oocyte.
66. Use according to any of paragraphs 43, 45-47, 49-52 or 55-65, wherein the germ cell is an ejaculated spermatozoon.
67. Use according to any of the preceding paragraphs, wherein the germ cell is an oocyte.
68. Use according to any of the preceding paragraphs, wherein the germ cell is an immature oocyte retrieved by aspiration or an immature oocyte retrieved by aspiration of small or medium sized antral follicles.
69. Use according to any of the preceding paragraphs, wherein the germ cell is an immature oocyte obtained after culture of at least one immature follicle at the primordial to the preantral stage.
70. Use according to any of the preceding paragraphs, wherein the mammalian gonadal cell is an immature oocyte with its adhering cumulus cells and/or an isolated immature oocyte retrieved from large antral follicles or an oocyte with its adhering cumulus cells and/or an immature oocyte retrieved from pre-antral follicles, small or medium sized antral follicles, a small human antral follicle being 0.4-<5 mm in diameter, a medium sized human antral follicle being 5-<15 mm and a large human antral follicle being 15 mm or above in diameter.
71. Use according to any of the preceding paragraphs, wherein the gonadal cell is an immature or mature oocyte obtained after culture of at least one immature follicle from a stage between the primordial and the preantral stage.

1. A method for inhibiting the sterol de novo biosynthesis in the cells of the mammalian gonad and/or at least one mammalian germ cell and/or gamete and/or gonadal derived cell supporting germ cells with sterols, thereby increasing the developmental competence of at least one mammalian gamete, zygote, early embryo, implanted blastocyst and/or embryo, and/or increasing the number of mammalian gametes with fertilisation capability, resulting in an increased number of zygotes, early embryos, implanted blastocysts and/or embryos per reproductive cycle in the female after mating, the method comprising administering a compound or a combination of compounds capable of inhibiting the sterol de novo biosynthesis of a mammal in need thereof; or for increasing the sterol efflux from at least one mammalian ovary and/or mammalian oocyte and/or mammalian female gamete and/or ovary derived cell surrounding an oocyte, thereby increasing the developmental competence of said at least one mammalian ovary and/or mammalian oocyte and/or mammalian female gamete and/or ovary derived cell surrounding an oocyte, the method comprising administering a compound or a combination of compounds capable of promoting the sterol efflux to a mammal in need thereof, or for increasing the developmental competence of at least one mammalian gamete, zygote, early embryo, implanted blastocyst and/or embryo and/or for increasing the number of mammalian gametes with fertilisation capability, resulting in an increased number of zygotes, early embryos, implanted blastocysts and/or embryos per reproductive cycle in the female, the method comprising adding a compound or a combination of compounds capable of inhibiting the sterol de novo biosynthesis of said at least one mammalian gamete, zygote, early embryo, implanted blastocyst and/or embryo to an in vitro medium which supports the respiration and/or maturation of at least one oocyte and/or at least one spermatozoon, or for increasing the sterol efflux from at least one mammalian ovary, oocyte, female gamete, and/or ovary derived cell surrounding an oocyte thereby increasing the developmental competence of said at least one mammalian ovary and/or mammalian oocyte and/or mammalian female gamete and/or ovary derived cell surrounding an oocyte, the method comprising adding a compound or a combination of compounds capable of promoting the sterol efflux to an in vitro medium which supports the respiration and/or maturation of at least one oocyte.

2. (canceled)

3. The method according to claim 1, wherein the mammal in the need thereof is female and the inhibition and/or efflux takes place during a period of 0-12 days before ovulation and/or 0-3 days after ovulation, or wherein the mammal in the need thereof is male and the inhibition takes place during a period of 0-12 days before ejaculation.

4. (canceled)

5. (canceled)

6. (canceled)

7. The method according to claim 1, wherein the compound is a substance or a combination of substances that antagonises one or more of the mammalian enzymes involved in the biosynthesis pathway between acetyl-coenzyme A and lanosterol, the enzymes being HMG-CoA reductase, mevalonate kinase, phosphomevalonate kinase, pyrophosphomevalonate decarboxylase, isopentenyl pyrophosphate isomerase, dimethylallyl transferase, geranyl transferase, squalene synthase, squalene monoxygenase, oxidosqualene lanosterol cyclase and lanosterol synthase, wherein the compound is a substance or a combination of substances that antagonises the 3-hydroxy-3-methylglutaryl coenzyme A reductase enzyme (HMG-CoA reductase), and/or wherein the compound is selected from the group consisting of cyclodextrin, chemically modified cyclodextrins, high densityliproprotein (HDL), an apoprotein derived from HDL, and sterol carrier protein I and II, and/or wherein the compound is a statin or a combination of statins, wherein the statin is selected from the group consisting of atorvastatin, cerivastatin, fluvastatin, BAY W 62, pravastatin, lovastatin (formerly called mevinolin or monacolin K), 28, HR 780, pravastatin, simvastatin, compatin, methyl-compactin, mevastatin (formerly called campactin or ML-236B), ML-236A, ML-236C, dihydrocompactin, monacolin J, monacolin L, monacolin M, monacolin X, dihydromonacolin L, mevinolin, 3β-hydroxycompactin acid monosodium salt (pravastatin, CS-514), synvinolin, cerivastatin and nystatin/triamcin.

8. (canceled)

9. (canceled)

10. (canceled)

11. (canceled)

12. (canceled)

13. The method according to claim 1, wherein:
(i) the amount of statin and/or compound capable of promoting the sterol efflux administered is between 0.01-100 mg per kg body weight per day; and/or,
(ii) the amount of statin and/or compound capable of promoting the sterol efflux administered to an in vitro medium is 0.01-1000 μM; and/or,
(iii) the culturing of at least one oocyte in an in vitro medium is between 1 second and 15 days; and/or,
(iv) the culturing of a spermatozoa in an in vitro medium is between 1 second and 8 days; and/or,
(v) the administration and/or addition is combined with an administration and/or addition of a gonadotrophin, wherein the amount of gonadotrophin administered in vitro is optionally between 0-1000 IU/L or between 0-5 IU FSH per kg body weight per day and 0-200 IU LH per kg body weight per day; and/or,
(vi) the administration and/or addition is combined with an administration and/or addition of a mammalian growth factor; and/or,
(vii) the mammal is selected from the group consisting of human, horse, cow, rat, mouse, pig, sheep, goat, llama, dog, cat and mink; and/or,
(viii) the mammalian gonadal cell is a human cell that support a germ cell with sterols or an isolated germ cell; and/or,
(ix) the mammalian gonadal cell is an cumulus enclosed oocyte or an isolated oocyte; and/or,
(x) the germ cell is an ejaculated spermatozoa; and/or,

14-24. (canceled)

25. A method according to claim 1, wherein the mammalian gonadal cell is an immature oocyte with its adhering cumulus cells and/or an isolated immature oocyte retrieved from large antral follicles or an oocyte with its adhering cumulus cells and/or an immature oocyte retrieved from pre-antral follicles, small or medium sized antral follicles, a small human antral follicle being 0.4-<5 mm in diameter, a medium sized human antral follicle being 5-<15 mm and a large human antral follicle being 15 mm or above in diameter.

26. A method according to claim 1, wherein the gonadal cell is an immature or mature oocyte obtained after culture of at least one immature follicle from a stage between the primordial and the preantral stage.

27. A cell culture medium comprising:
(a) a statin or a combination of statins;
(b) a compound or combination of compounds capable of promoting the sterol efflux, such as a cyclodextrin; or,
(c) a statin and a cyclodextrin.

28. (canceled)

29. (canceled)

30. The cell culture medium claim 27, wherein said medium is a chemically defined cell culture medium.

31. The cell culture medium claim 27, further comprising at least one growth factor.

32. The cell culture medium claim 27, wherein said growth factor is selected from the group consisting of gonadotropin (FSH, LH, CG), IGF, EGF, Insulin, growth hormone, interleukines and other peptid factors.

33. A method for maturating an immature oocyte obtained from a stage between the primordial follicle and the pre-antral stage comprising culturing said immature oocyte in a cell culture medium according to claim 27.

34. A pharmaceutical composition comprising:
(a) a combination of a compound capable of inhibiting the sterol de novo biosynthesis and compound capable of increasing the sterol efflux in a combination together with a pharmaceutically acceptable carrier wherein said compounds are optionally a statin and a cyclodextrin;
(b) a combination of a compound capable of inhibiting the sterol de novo biosynthesis and a growth factor, wherein said compound is optionally a statin; or,
(c) a combination of a compound capable of promoting the sterol efflux and a growth factor, wherein said compound is optionally a cyclodextrin;
and wherein the composition optionally further comprises a growth factor selected from the group consisting gonadotropin (FSH, LH, CG), IGF, EGF, Insulin, growth hormone, interleukines and other peptid factors.

35-42. (canceled)

43. A method of using a compound of claim 1, or a combination of compounds of claim 1, capable of inhibiting the sterol de novo biosynthesis in mammalian gonads and/or at least one mammalian germ cell and/or gamete and/or gonadal derived cell, for the preparation of a medicament for inhibiting the sterol de novo biosynthesis in at least one mammalian germ cell and/or gamete and/or gonadal derived cell supporting germ cells with sterols, thereby increasing the developmental competence of at least one mammalian gamete, zygote, early embryo, implanted blastocyst and/or embryo, and/or for increasing the number of mammalian gametes with fertilisation capacity, resulting in an increased number of zygotes, early embryos, implanted blastocysts and/or embryos per reproductive cycle in a female after mating.

44. A method of using a compound of claim 1, or a combination of compounds of claim 1, capable of promoting the sterol efflux from at least one mammalian ovary and/or mammalian oocyte and/or mammalian female gamete and/or ovary derived cell surrounding an oocyte, for the preparation of a medicament for promoting the sterol efflux in at least one mammalian ovary and/or mammalian oocyte and/or mammalian female gamete and/or ovary derived cell surrounding an oocyte, thereby increasing the developmental competence of at least one mammalian ovary and/or mammalian oocyte and/or mammalian female gamete and/or ovary derived cell surrounding an oocyte.

45-71. (canceled)
